(19) United States
(12) Patent Application Publication
(10) Pub. No.: US 2019/0345459 A1
(43) Pub. Date: Nov. 14, 2019

(54) ENZYME COMPLEX FOR LIGNOCELLULOSIC MATERIAL DEGRADATION

(71) Applicant: Yeda Research and Development Co. Ltd., Rehovot (IL)

(72) Inventors: Edward A. BAYER, Ramot-HaShavim (IL); Lital DAVIDI, Rehovot (IL); Sarah MORAIS, Rehovot (IL); Lior ARTZI, Rehovot (IL); Yonathan ARFI, Rehovot (IL)

(73) Assignee: Yeda Research and Development Co. Ltd., Rehovot (IL)

(21) Appl. No.: 16/317,842

(22) PCT Filed: Aug. 30, 2017
(86) PCT No.: PCT/IL2017/050970

§ 371 Date: Jan. 15, 2019

(30) Foreign Application Priority Data
Aug. 30, 2016 (IL) 247569

(51) Int. Cl. C12N 9/02; C12N 9/08; C12P 19/00

(52) U.S. Cl. CPC C12N 9/0061; C12Y 110/03002; C12Y 111/01014; C12P 19/00; C12Y 111/01016; C12N 9/0065; C12Y 111/01013

(57) ABSTRACT

A lignocellulolytic multi-enzyme complex in the form of a cellulosome, which includes a lignin-modifying enzyme and a carbohydrate-active enzyme, is provided herewith, as well as bifunctional chimeric enzymes having lignin and cellulose/hemicellulose degrading capacity. Also provided are methods of degrading lignocellulolytic biomass, and compositions and systems for effecting the same.

### FIELD AND BACKGROUND OF THE INVENTION

The present invention, in some embodiments thereof, relates to biomass-degrading enzyme complexes, and more particularly, but not exclusively, to artificial cellulosomes designed for efficient degradation of lignocellulosic biomass into useful products.

Plant biomass is one of the most abundant and renewable sources of organic material on earth. Given its widespread availability and renewability, it is considered a promising resource for alternative and sustainable energy production. Plant cell wall comprises lignocellulose, a heterogeneous amalgamation of cellulose, hemicellulose and lignin. Total degradation of biomass can therefore be seen as breakdown of cellulose, hemicellulose and lignin, preferably into useful degradation products.

Cellulose and hemicellulose are attractive components for the production of biofuels or synthons, as these polysaccharides can be biologically hydrolyzed to simple sugars, which in turn can be converted into ethanol or other high-value chemicals. A group of enzymes known as cellulases performs hydrolysis of cellulose. They are classically divided into several groups: 1) exoglucanases, which can only cleave at the ends of the linear cellulose chain sequentially (2-4 glucose units at a time), and accordingly possess a tunnel-like active site; 2) endoglucanases, which cleave the cellulose chain in the middle (exposing new individual chain ends), commonly possess a groove, or cleft, which can fit any part of the linear chain; and 3) processive endoglucanases, considered as an intermediate group which, like endoglucanases, can cleave the cellulose chain in the middle but after the initial cleavage, can continue to sequentially degrade the cellulose chain like exoglucanases. Another classical group is β-glucosidases, which hydrolyze the terminal non-reducing β-D-glucose residues of cellodextrins (in particular cellobiose, which is one of the major end products of cellulose degradation) into monosaccharides.

Hemicellulose is degraded by a group of enzymes known as hemicellulases, that can be divided into two main types: those that cleave the main chain backbone (xylanases, which cleave randomly the β-1,4 linkage of xylan to produce xyloligosaccharides, which are further hydrolyzed into xylose by β-1,4 xylosidases); and those that degrade side chain substituents or short end products (such as arabinofuranosidase and acetyl esterases). Both type of enzymes (cellulases and hemicellulases) are needed in order to achieve complete plant cell wall degradation.

Lignin is mostly considered a hindrance in bioethanol production processes. Indeed, although lignin-derived aromatic compounds are valuable in the green chemistry sector, this complex organic heteropolymer encases the cellulose/hemicellulose fibers and thus limits the accessibility of enzymes or chemicals. As a result, and in order to increase the amount of fermentable sugars produced from plant biomass, lignin must be removed. The currently available techniques to remove lignin from the biomass are ineffective. For example, chemical pretreatment of biomass, with acids or organic solvents, prior to enzymatic digestion, generates lignocellulose-derived by-products such as phenolic compounds that inhibit the enzymatic biocatalysts. More favorable techniques, based on the use of lignin-degrading enzymes, such as laccase and lignin peroxidase, have long been identified; however, their utilization in the biofuel production process is currently limited.

Laccases (EC 1.10.3.2) are well-studied blue multi-copper oxidases (type 1) found in fungi, plants and microorganisms, that catalyze the oxidation of a wide variety of phenolic and non-phenolic compounds, with concomitant reduction of molecular oxygen to water. The broad substrate range of laccases allows their use in numerous types of industrial and biotechnological fields, such as the paper, textile and food industries. Known functions of fungal and bacterial laccases include roles in morphogenesis, sporulation and pigmentation. Both bacterial and fungal forms can effect lignin lignocellulose degradation, whereas plant laccases are involved in lignin synthesis in the plant cell wall.

A laccase-like enzyme (Tfu_1114) was characterized in the model cellulolytic bacterium Thermobifida fusca [Chen, C-Y et al., Appl Microbiol Biotechnol, 2013, 97, pp. 8977-8986]. Tfu_1114 is able to catalyze the oxidation of several phenolic and non-phenolic lignin-related compounds, such as 2,6-dimethoxyphenol (2,6-DMP), veratryl alcohol and guaiacol. However, this laccase exhibits only one copper atom per protein instead of the four found in canonical laccases. It was shown that Tfu_1114 can significantly increase the hydrolysis of bagasse polysaccharides when combined with a cellulase or a xylanase from the same organism. This enhancement is linked to the oxidation of phenolic subunits of lignin, and the generation of free radicals, which could create cleavage sites in the network structure of the lignin-carbohydrate complex, consequently exposing more polysaccharide fibers to the glycoside hydrolases.

The cellulolytic enzymes produced by T. fusca, which degrade lignocellulose, are secreted individually as free enzymes, like in the large majority of aerobic microorganisms. By contrast, a restricted number of anaerobic bacteria perform efficient degradation of plant biomass through the secretion of unique, large multi-enzyme complexes termed “cellulosomes”. These complexes are based on a non-catalytic subunit called scaffoldin that binds to the insoluble substrate via a cellulose-specific carbohydrate-binding module (CBM). The scaffoldin also contains a set of subunit-binding modules termed “cohesins” that mediate the specific incorporation and organization of catalytic subunits through the dockerin, a complementary binding module carried by each enzymatic subunit. The existence of cellulosome complexes in anaerobic fungi, which differs by the absence of canonical cohesins and dockerins, has also been reported.

Previous studies have demonstrated the high efficiency of cellulosomes for the degradation of cellulose and hemicellulose, due to the spatial proximity of synergistically acting enzymes and to the locally increased concentrations in enzymes and substrate. However, cellulosomal systems discovered to date all appear to be lacking some of the key oxidative enzymes produced by the majority of aerobic lignocellulolytic microbes, involved in the reduction of cellulose crystallinity and in lignin breakdown.

Two decades ago, the concept of designer cellulosome was proposed, based on the modular nature of the cellulosome complex. Designer cellulosomes were devised as a tool to manipulate cellulosomal architecture and to incorporate non-cellulosomal enzymes into these artificial complexes [Bayer, E A. et al., Trends Biotechnol, 1994, 12, pp. 379-86]. This is accomplished by taking advantage of the high specificity between cohesins and dockerins originating from the same species. A chimeric scaffoldin, bearing several cohesin modules from multiple origins, can thus be designed, as well as chimeric enzymes, bearing the corresponding dockerin, thus enabling self-assembly of a tailor-made enzymatic complex. Thus, a typical designer cellulosome includes a chimaeric scaffoldin containing a CBM and several cohesin modules derived from different species, having divergent specificities. The complex further includes plant cell wall-degrading enzymes, each having a complementary and specific dockerin module that mediates selective binding to one of the divergent cohesins. The chimaeric scaffoldin enables the control of the location of each cellulolytic enzyme in the cellulosomal complex as well as its molar ratio. This Lego-like complex has been used to test the effects of enzymatic compositions, relative positioning of the enzymes within the complex, and their spatial proximity, which together generate the synergistic action of the cellulosomal components [Mingardon, F. et al., Appl Environ Microbiol, 2007, 73, pp. 7138-7149; and Stern, J. et al., PLoS One, 2015, 10, e0127326].

Previous reports using designer scaffoldins resulted in enhanced activity of various recalcitrant substrates degradation [Caspi, J. et al., Journal of Biotechnology, 2008, 135, pp. 351-357; Caspi, J. et al., Applied and Environmental Microbiology, 2009, 75, pp. 7335-7342; Moraïs, S. et al., mBio, 2010, 1, e00285-10; and Moraïs, S. et al., mBio, 2011, 2, e00233-11]. In most of these, configuration of designer cellulosomes mimicked the overall simple architecture of C. thermocellum. More complex structures have also been described [Mingardon et al., Applied and Environmental Microbiology, 2007, 73, pp. 7138-7149].

One of the largest forms of homogeneous artificial cellulosome reported to date contains a chimaeric scaffoldin with six divergent cohesins, integrating six dockerin-bearing cellulolytic enzymes (xylanases and cellulases) [Morais et al., MBio, 2012, 11, 3(6), pii: e00508-12. doi: 10.1128/mBio.00508-12].

International Patent Application Publication No. WO 1997/014789 discloses an enzymatic array, which composition comprises one or more enzymes non-covalently bound to a peptide backbone, wherein at least one of the enzymes is heterologous to the peptide backbone and the peptide backbone is capable of having bound thereto a plurality of enzymes. The array is reported as useful, for example, in recovery systems, targeted multi-enzyme delivery systems, soluble substrate modification, quantification type assays, and other applications in the food industry, feed, textiles, bioconversion, pulp and paper production, plant protection and pest control, wood preservatives, topical lotions and biomass conversions.

U.S. Patent Application Nos. 2010/057064 and 2011/0306105 disclose designer cellulosomes for efficient hydrolysis of cellulosic material and more particularly for the generating of ethanol.

International Patent Application Publication No. WO 2012/055863 discloses covalent cellulosomes and uses thereof; in particular, enzyme constructs with increased enzymatic activity based on the use of spacers interconnecting catalytic modules are disclosed, and polynucleic acids encoding these constructs.

Vazana and co-workers [Vazana, Y. et al., Biotechnol Biofuels, 2013, 6, 182] investigated the spatial organization of the scaffoldin subunit and its effect on cellulose hydrolysis by designing a combinatorial library of recombinant trivalent designer scaffoldins, which contain a carbohydrate-binding module (CBM) and three divergent cohesin modules.

Additional prior art documents include, for example, International Patent Application Publication No. WO 2010/096562, U.S. Patent Application Publication Nos. 2009/0220480, 2009/0155238, 2011/0016545, 2013/0189745, 2014/0030769, 2015/0167030 and 2016/0186156, and U.S. Pat. No. 9,034,609.

### SUMMARY OF THE INVENTION

The present invention provides an artificial enzyme complex in the form of a cellulosome that unlike naturally occurring cellulosomes and known artificial cellulosomes, exhibits the capacity to break down lignin. The presently disclosed cellulosome includes at least one lignin-degrading enzyme, such as laccase, which acts in synergy to break down lignocellulosic biomass, such as wheat straw, into sugars and other by-products. This artificial lignocellulolytic multi-enzyme complex can self-assemble in vivo and in vitro and be used in compositions and systems designed to degrade biomass.

According to an aspect of some embodiments of the present invention there is provided a lignocellulolytic multi-enzyme complex that includes at least one lignin-modifying enzyme (LME) and at least one carbohydrate-active enzyme (CAE).

According to some embodiments of the invention, the complex further includes a scaffold polypeptide, the scaffold polypeptide comprises at least one cohesin module, wherein the cohesin modules are separated by linkers that comprise 1-100 amino acids, and each of the lignin-modifying enzyme and the carbohydrate-active enzyme is having a dockerin module that matches at least one of the cohesin modules, the dockerin module is bound to the cohesin module.

According to some embodiments of the invention, the lignin-modifying enzyme is in a form of a chimeric enzyme that includes the lignin-modifying enzyme and a carbohydrate-active enzyme, each attached to the dockerin module via a linker that comprises 1-100 amino acids.

According to some embodiments of the invention, each of the dockerin modules matches a single cohesin module in the scaffold polypeptide.

According to some embodiments of the invention, the scaffold polypeptide further includes at least one substrate-binding module (SBM) attached to at least one of the cohesin modules via linkers that comprise 1-100 amino acids.

According to some embodiments of the invention, the substrate-binding module is derived from a bacterium selected from the group consisting of clostridial and related genera (notably Ruminiclostridium species), including Clostridium (Ruminiclostridium) thermocellum, Clostridium cellulolyticum, Clostridium cellulovorans, Clostridium clariflavum, Clostridium papyrosolvens, Clostridium josui, Clostridium alkalicellulosi, Bacteroides (Pseudobacteroides) cellulosolvens, Acetivibrio cellulolyticus, Caldicellulosiruptor (Anaerocellum) species, Caldicellulosiruptor bescii (Anaerocellum thermophilum), Caldicellulosiruptor saccharolyticus DSM 890, Caldicellulosiruptor obsidiansis, Caldicellulosiruptor lactoaceticus, Caldicellulosiruptor kronotskyensis and Caldicellulosiruptor kristjanssonii.

According to some embodiments of the invention, the lignin-modifying enzyme is selected from the group consisting of a laccase (EC 1.10.3.2), a lignin peroxidase (EC 1.11.1.14), a manganese peroxidase (EC 1.11.1.13) and a versatile peroxidase (EC 1.11.1.16).

According to some embodiments of the invention, the lignin-modifying enzyme is a laccase (EC 1.10.3.2).

According to some embodiments of the invention, the laccase is derived from a Thermobifida fusca.

According to some embodiments of the invention, the carbohydrate-active enzyme (CAE) is a cellulose- and/or hemicellulose-degrading enzyme.

According to some embodiments of the invention, carbohydrate-active enzyme (CAE) is selected from the group consisting of a cellulase, a hemicellulose, a glycoside hydrolase, an exoglucanase, an endoglucanase, a xylanase, an exoxylanase, an endoxylanase, a mannanase, an arabinase, an arabinofuranosidase, a xyloglucanase, a β-xylosidase, a β-glucosidase, a polysaccharide lyase, a mannosidase and carbohydrate esterase.

According to some embodiments of the invention, the carbohydrate-active enzyme is a cellulase and/or a hemicellulase classified in a glycoside hydrolase (GH) family selected from the group consisting of GH5, GH6, GH7, GH8, GH9, GH10, GH11, GH12, GH26, GH30, GH43, GH44, GH45, GH48, GH51, GH61, GH74, GH81, GH98 and GH124.

According to some embodiments of the invention, the endoglucanase is selected from the group consisting of GH5 EC 3.2.1.4, GH6 EC 3.2.1.4, GH8 EC 3.2.1.4, GH9 EC 3.2.1.4, GH9 EC 3.2.1.74, GH16 EC 3.2.1.39, GH16 EC 3.2.1.6, GH44 EC 3.2.1.4, GH81 EC 3.2.1.39 and GH124 EC 3.2.1.4.

According to some embodiments of the invention, the exoglucanase is selected from the group consisting of GH48 EC 3.2.1.91 and GH6 EC 3.2.1.176.

According to some embodiments of the invention, endoxylanase is selected from the group consisting of GH10 EC 3.2.1.8, GH11 EC 3.2.1.8, GH11EC 3.2.1.32, GH30 EC 3.2.1.8, GH43 EC 3.2.1.8, GH98 EC 3.2.1.8.

According to some embodiments of the invention, the complex comprising at least one cellulase and at least one hemicellulase.

According to some embodiments of the invention, the carbohydrate-active enzyme is derived from a bacterium selected from the group consisting of Thermobifida fusca, Clostridium thermocellum, Geobacillus stearothermophilus, Clostridium clariflavum, Clostridium (Ruminiclostridium) thermocellum, Clostridium cellulolyticum, Clostridium cellulovorans, Clostridium clariflavum, Clostridium papyrosolvens, Clostridium josui, Clostridium alkalicellulosi, Bacteroides (Pseudobacteroides) cellulosolvens, Acetivibrio cellulolyticus, Caldicellulosiruptor bescii (Anaerocellum thermophilum), Caldicellulosiruptor saccharolyticus DSM 890, Caldicellulosiruptor obsidiansis, Caldicellulosiruptor lactoaceticus, Caldicellulosiruptor kronotskyensis, Caldicellulosiruptor kristjanssonii, Ruminococcus albus, Ruminococcus bromii, Ruminococcus champanellensis and Ruminococcus flavefaciens.

According to some embodiments of the invention, each of the dockerin module and/or the cohesin module is individually derived [originate] from a bacterium selected from the group consisting of Acetivibrio cellulolyticus, Archaeoglobus fulgidus, Bacteroides cellulosolvens, pseudo-Bacteroides cellulosolvens, Clostidium alkalicellulosi, Clostridium acetobutylicum, Clostridium bornimense, Clostridium cellobioparum, Clostridium cellulolyticum, Clostridium cellulovorans, Clostridium clariflavum, Clostridium josui, Clostridium papyrosolvens, Clostridium perfringens, Clostridium saccharoperbutylacetonicum, Clostridium sp. BNL1100, Clostridium straminisolvens, Clostridium termitidis, Clostridium thermocellum, Ruminococcus albus, Ruminococcus bromii, Ruminococcus champanellensis and Ruminococcus flavefaciens.

According to an aspect of some embodiments of the present invention there is provided a chimeric enzyme that includes a lignin-modifying enzyme (LME), a carbohydrate-active enzyme (CAE) and a dockerin module.

According to some embodiments of the invention, each of the lignin-modifying enzyme and the carbohydrate-active enzyme is attached to the dockerin module via a linker of 1-100 amino acids.

According to some embodiments of the invention, the lignin-modifying enzyme is selected from the group consisting of a laccase (EC 1.10.3.2), a lignin peroxidase (EC 1.11.1.14), a manganese peroxidase (EC 1.11.1.13) and a versatile peroxidase (EC 1.11.1.16).

According to some embodiments of the invention, the lignin-modifying enzyme is a laccase.

According to some embodiments of the invention, the laccase derived from a Thermobifida fusca.

According to some embodiments of the invention, the carbohydrate-active enzyme is selected from the group consisting of a cellulase, a hemicellulose, a glycoside hydrolase, an exoglucanase, an endoglucanase, a xylanase, an exoxylanase, an endoxylanase, a mannanase, an arabinase, an arabinofuranosidase, a xyloglucanase, a β-xylosidase, a β-glucosidase, a polysaccharide lyase, a mannosidase and carbohydrate esterase.

According to some embodiments of the invention, the carbohydrate-active enzyme is a cellulase classified in a glycoside hydrolase (GH) family selected from the group consisting of GH5, GH6, GH7, GH8, GH9, GH10, GH11, GH12, GH26, GH30, GH43, GH44, GH45, GH48, GH51, GH61, GH74, GH81, GH98 and GH124.

According to some embodiments of the invention, the carbohydrate-active enzyme is a xylanase.

According to some embodiments of the invention, the xylanase is XynT6 derived from Geobacillus stearothermophilus.

According to some embodiments of the invention, the chimeric enzyme further includes a tag selected from the group consisting of a solubilisation tag, an affinity binding tag, a detection tag, a fluorescence tag a chromatography tag, an epitope tag, a protein purification tag and a protein tag.

According to some embodiments of the invention, the chimeric enzyme is having SEQ ID No. 1.

According to some embodiments of the invention, the chimeric enzyme further includes at least one substrate-binding module.

According to some embodiments of the invention, the substrate-binding module in the chimeric enzyme is a cellulose-binding module.

According to some embodiments of the invention, the cellulose-binding module is a cellulose-binding domain (CBD).

According to an aspect of some embodiments of the present invention there is provided a composition for degrading a cellulosic or lignocellulosic material, which includes the lignocellulolytic multi-enzyme complex presented herein.

According to an aspect of some embodiments of the present invention there is provided a system for degrading a cellulosic or lignocellulosic material, the system includes the lignocellulolytic multi-enzyme complex presented herein or the composition comprising the same.

According to an aspect of some embodiments of the present invention there is provided a method for degrading a cellulosic or lignocellulosic material, the method is effected by exposing the cellulosic or lignocellulosic material to the lignocellulolytic multi-enzyme complex presented herein.

According to an aspect of some embodiments of the present invention there is provided a genetically modified host cell that includes polynucleotides encoding the plurality of components of the lignocellulolytic multi-enzyme complex presented herein.

According to some embodiments of the invention, the polynucleotides in the host cell are having a sequence selected from the group consisting of SEQ ID Nos. 11-17.

According to an aspect of some embodiments of the present invention there is provided a genetically modified host cell that includes a polynucleotide encoding the chimeric enzyme presented herein.

According to some embodiments of the invention, the polynucleotide in the host cell is having SEQ ID No. 11.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a certain property or characteristic, refer to a process, a composition, a structure or an article that is totally devoid of the property or characteristic or characterized by less than about 5, 1, 0.5 or 0.1 percent of the property or characteristic, compared to a given standard.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

As used herein, the term “plurality” indicates at least two.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

It is expected that during the life of a patent maturing from this application many relevant enzyme complexes capable of degrading lignocellulosic biomass will be developed and the scope of the phrase “lignocellulolytic multi-enzyme complex” is intended to include all such new technologies a priori.

### BRIEF DESCRIPTION OF THE SEVERAL VIEWS OF THE DRAWINGS

Some embodiments of the invention are herein described, by way of example only, with reference to the accompanying drawings or images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents a schematic illustration of the recombinant proteins used in the example presented herein, wherein the numbers 5, 11, and 48 refer to the corresponding GH family (GH5, GH48, GH11) of the catalytic module; uppercase characters (A, C, F, T) indicate the source of the cohesin module and lowercase characters (a, c, f, t) indicate the source of the dockerin module;

FIG. 2 presents comparative plot of xylanase activity of XynT6, Xyn-c and Xyn-c-Lac (SEQ ID No. 1) on beechwood xylan at 50° C., wherein the assay was repeated twice and the error bars indicate the standard deviation from the mean of triplicate samples from one experiment;

FIG. 3 presents ELISA assay results obtained for cohesin-dockerin binding, wherein Xyn-c and Xyn-c-Lac (SEQ ID No. 1) interacted with the cohesin 1 from C. cellulolyticum CipC, and wherein Xyn-c-Lac (SEQ ID No. 1) failed to interact with C. thermocellum cohesin 3 from CipA as a negative control (error bars indicate the standard deviation from the mean of triplicate samples from one experiment);

FIG. 4 presents comparative activity plot of the wild-type laccase (Lac) and chimeric Xyn-c-Lac (SEQ ID No. 1) towards different substrates;

FIG. 5 presents an SDS-PAGE gel slab of the bound and unbound fractions of the various designer cellulosome preparations, according to some embodiments of the present invention, showing the results of the affinity pull-down assay serving for the assessment of cellulosome complex formation, wherein dockerin-bearing enzymes interacting properly with matching cohesins of the scaffoldin protein appear as bands in the bound fraction (marked with arrows), and none visible bands in the unbound fraction indicate enzymes that failed to interact properly with the matching cohesins of the scaffoldin;

FIGS. 6A-B present the results of an electrophoresis mobility experiment to verify the formation of a designer cellulosome complex, according to some embodiments of the present invention, wherein FIG. 6A is an SDS-PAGE gel slab and FIG. 6B is a non-denaturing gel slab;

FIG. 7 presents a bar plot showing the comparative degradation of non-treated wheat straw incubated for 72 hours at 50° C. with laccase in tetravalent designer cellulosomes and free-enzyme combinations, wherein bar 1 represents substrate degradation by bifunctional chimaeric xylanase-tagged laccase (Xyn-c-Lac (SEQ ID No. 1)), bar 2 represents substrate degradation by xylanase tag alone (Xyn-c), bar 3 represents substrate degradation by three free dockerin-bearing GHs (Xyn11V-a (SEQ ID No. 3), t-48A (SEQ ID No. 4) and f-5A (SEQ ID No. 2)), bar 4 represents substrate degradation by the three free GHs with the additional xylanase tag (Xyn-c), bar 5 represents substrate degradation by the three free GHs with the addition of the xylanase-tagged laccase (Xyn-c-Lac; SEQ ID No. 1), bar 6 represents substrate degradation by the three scaffoldin-complexed GHs, bar 7 represents substrate degradation by scaffoldin-complexed GHs+xylanase tag, and bar 8 represents substrate degradation by scaffoldin complexed GHs+laccase, wherein each reaction was performed three times and error bars represent standard deviations;

FIG. 8 presents kinetics studies over 7 days of the tetravalent designer cellulosome, according to some embodiments of the present invention, bearing the Xyn-c-Lac (SEQ ID No. 1) enzyme as compared to selected controls; and

FIG. 9 presents a plot of comparative enzymatic activity of designer cellulosomes with an addition of the free Xyn-c-Lac (marked “A”), or containing the Xyn-c-Lac, according to some embodiments of the present invention (marked “B”) on degradation of brewer's spent grain and apple pomace.

### DESCRIPTION OF SPECIFIC EMBODIMENTS OF THE INVENTION

The present invention, in some embodiments thereof, relates to biomass-degrading enzyme complexes, and more particularly, but not exclusively, to artificial cellulosomes designed for efficient degradation of lignocellulosic biomass into useful products.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As discussed hereinabove, the complete degradation of cellulosic biomass is hindered by the presence and/or the inefficient degradation of lignin. The production of lignocellulosic biofuel initially involves the deconstruction of cell wall polymers into simple sugars; however, lignin acts as a physical barrier that restrict the access of hydrolytic enzymes to cellulose and hemicellulose components of the plant cell wall. As further discussed herein, naturally occurring and current designer cellulosomes do not provide a solution to the problems associated with lignin and total degradation of cellulosic biomass.

While searching for a solution to the problem of total biomass degradation, the present inventors have contemplated the use of an enzyme that catalyses or facilitates the degradation of lignin, thereby contribute to current efforts to overcome limitations of enzymatic degradation of lignocellulosic biomass. The present inventors have contemplated the integration of such non-cellulosomal enzyme that degrades lignin in the context of a designer cellulosome complex with the intention of harnessing the proximity effect of ordered hydrolytic enzymes to maximize their combined synergistic effects, whereas the gist of this concept was to afford total degradation of lignocellulose.

While reducing the present invention to practice, the present inventors examined the possibility of converting a laccase or a laccase-like enzyme, such as Tfu_1114, to the cellulosomal mode by fusion of the enzyme to a dockerin module; however, unlike manipulations of cellulosomal enzymes, the insertion of non-cellulosomal enzymes into designer cellulosome complex is far from being trivial, particularly a protein that contains a single catalytic module, and indeed the exemplary laccase-like enzyme Tfu_1114 tethered to a dockerin module was poorly expressed (c-Lac (SEQ ID No. 6) and MBP-tev-c-Lac (SEQ ID No. 7). Surprisingly, a dockerin-xylanase chimera (referred to herein as “Xyn-c-Lac”; an example embodiment thereof is assigned SEQ ID No. 1) was capable of overexpression as an active lignin-oxidase, while the impact of the conversion on its lignin-oxidase activity was negligible. As demonstrated in the Examples section that follows below, this chimaeric bifunctional enzyme was incorporated into a designer cellulosome successfully alongside with two cellulases and a xylanase and used to decompose a wheat straw substrate. The results indicated that the simultaneous degradation by cellulosome action of the three components of lignocellulose, i.e., cellulose, hemicellulose and lignin, afforded a highly effective and efficient designer cellulosome that can produce about twice the amount of usable sugars from wheat straw compared to other cellulosome or to mixtures of free enzymes.

The successful incorporation of a non-cellulosomal lignin-degrading enzyme into a designer cellulosome offers new potential for enzymatic degradation of lignocellulosic biomass and can contribute to future efforts for production of alternative fuels. The previously reported incorporation of enzymes into designer cellulosomes such as β-glucosidases, LPMOs and expansins has proved an efficient strategy for enhancement of biomass degradation by glycosides hydrolases (GH; EC 3.2.1.-). These accessory enzymes acted in concert with glycoside hydrolases, either by releasing product inhibition or by acting directly on the substrate. Designer cellulosomes that have contained either copper-dependent redox enzymes, called lytic polysaccharide monoxoygenases (LPMOs), or the laccase have enabled combination of both hydrolytic and oxidative activities in the same reaction, which would not have been possible in natural cellulosomes that are restricted to anaerobic environments.

The non-cellulosomal lignin-degrading enzyme presented herein is now part of the miniature toolbox that can serve to enhance the value of designer cellulosome technology and contribute synergistically to biomass degradation, either by assisting the glycoside hydrolases in their methodic degradation of polysaccharide substrates or by attacking different components of the plant cell-wall matrix.

Lignocellulolytic Multi-Enzyme Complex:

According to an aspect of some embodiments of the present invention, there is provided a lignocellulolytic multi-enzyme complex that includes at least one lignin-modifying enzyme (LME) and at least one carbohydrate-active enzyme (CAE).

According to some embodiments, the lignin-modifying enzyme does not occur in nature as a cellulosomal enzyme, or in other words, the lignin-modifying enzyme is a non-cellulosomal enzyme, and thus any cellulosome, or any multi-enzyme complex, that exhibit a lignin-modifying enzyme, is by definition an artificial multi-enzyme complex or an artificial cellulosome. The term “cellulosomal enzyme” refers to an enzyme that in nature is typically found as part of a cellulosome complex. A cellulosomal enzyme typically contains the means to participate in the formation of a cellulosome, namely a dockerin module, which forms a part of the polypeptide chain in its naturally occurring state as an inherent part of the protein. The term “non-cellulosomal enzyme” refers to an enzyme that in nature is active as a free enzyme, typically secreted into the environment. Such enzymes usually do not have a dockerin module.

As used herein, the term “enzyme” refers to a polypeptide having a catalytic activity towards a certain substrate or substrates. The term “artificial”, as used herein in the context of the multi-enzyme complex of the present invention, indicates that the complex is artificially/synthetically made, and does not occur in nature. It is to be understood that naturally occurring cellulosome complexes are excluded from the scope of the present invention.

The terms “polypeptide,” “peptide” and “protein” are used interchangeably herein to refer to a polymer of amino acid residues. The terms “polynucleotide” or “oligonucleotide” are used interchangeably herein to refer to a polymer of nucleic acids.

The term “module”, as used herein, refers to a part of a macromolecule that exhibits a particular biologic activity, such as catalytic activity, binding and the like. The term “module” encompasses polypeptides, protein domains and non-proteinaceous entities, such as nucleic acid oligomers, polysaccharides, fatty acids and any combination thereof. The term “domain”, as used herein, may refer to a proteinaceous module.

The term “complex” as used herein refers to a coordination or association of components linked preferably by non-covalent interactions, or by covalent bonds. In the context of embodiments of the present invention, a complex comprises macromolecular components that exhibit an affinity to one-another via specific structure recognition sites, which drives the component to bind to one-another reversibly. The term “complex” is not meant to encompass macromolecular entities of a single polypeptide chain that exhibit more than one domain and thus more than one biochemical activity. In some embodiments, the term “complex” is used to define an association of macromolecular components that self-assemble reversibly and reproducibly into a super-structure. Thus, in the context of some embodiments of the present invention, the complex can form spontaneously from its macromolecular components by virtue of the affinity and specificity of the binding modules that occur in its components. For example, the complex will form in vivo in a host cell that expresses the components of the complex, or in its secretions and lysate. Similarly, the complex will form in vitro when all its components will be placed in the same media.

The term “multi-enzyme complex” as used herein indicates a complex comprising a plurality of enzymes, namely, at least two enzymes and preferably more. The multi-enzyme complex of the present invention further includes non-catalytic components, such as structural components, specific binding components and substrate-binding components. In the context of some embodiments of the present invention, the lignocellulolytic multi-enzyme complex presented herein is an artificial cellulosome.

Lignin-Modifying Enzymes (LMEs):

In the context of embodiments of the present invention, a lignin-modifying enzyme is any enzyme that catalyses the breakdown (degradation) of lignin, thereby enhancing accessibility of the polysaccharides to the carbohydrate-active enzymes, which results in increasing the enzymatic release of soluble sugars from lignocellulose.

According to some embodiments, lignin-modifying enzymes are lignin-oxidases, which constitute a group of enzymes that share a common catalytic activity on lignin, namely the breakdown of lignin by a catalytic oxidative reaction. Indeed, most lignin-modifying enzymes are not hydrolytic, but rather oxidative (electron withdrawing) by their enzymatic mechanisms (oxidases). In the context of embodiments of the present invention, LMEs include peroxidases, such as lignin peroxidase (EC 1.11.1.14), manganese peroxidase (EC 1.11.1.13), versatile peroxidase (EC 1.11.1.16), phenoloxidases of the laccase type (EC 1.10.3.2) and laccase-like enzymes.

In the context of embodiments of the present invention, the lignin-modifying enzyme can originate from any organism, plant, fungi or bacteria, without limitation. According to some embodiments of the invention, the LME is a variant of a naturally occurring enzyme, engineered specifically to exhibit properties that render it suitable for forming a part of the lignocellulolytic multi-enzyme complex presented herein.

The terms “variant”, “derivative”, “genetically engineered” and “modified” are used interchangeably to describe a polypeptide which differs from a wild-type amino acid sequence by one or more amino acid substitutions introduced into the sequence, and/or one or more deletions/additions; typically, a naturally occurring variation is referred to as a mutant, while an artificially engineered mutation leads to a variant. As used herein, the term “wild type” refers to the naturally occurring DNA/protein. It is to be understood that a derivative/variant generally retains the properties or activity observed in the wild-type to the extent that the derivative is useful for similar purposes as the wild-type form. For example, when the terms refer to an enzyme, they indicate that the wild-type sequence has been modified substantially without adversely affecting its catalytic activity (its ability to recognize a substrate and catalyse a chemical transformation in the substrate) in a manner that is substantially comparable to the wild-type enzyme. Typically, the catalytic domain is maintained. For another example, when the terms refer to the matching cohesin/dockerin, respectively, they indicate that the wild-type sequence has been modified without adversely affecting its ability to recognize the matching cohesin/dockerin, respectively. Typically, the recognition site of the relevant counterpart, also referred to as the binding site, is maintained. When referring to any protein in the context of the present invention as being “derived from” a wild-type protein or a certain organism, it is meant that it is a variant of the wild-type protein that “originates from” the specified organism.

In some embodiments of the present invention, the lignin-modifying enzyme is a laccase or a laccase-like enzyme. Laccases constitute a group of multi-copper proteins of low specificity acting on both o- and p-quinols, and often acting on aminophenols and phenylenediamine. A laccase-like enzyme shares the same activity, but may exhibit a single copper ion cofactor.

Under the widely accepted terminology used by the Carbohydrate-Active Enzymes (CAZy) database (www.cazy.org), laccase is a member of Auxiliary Activity Family 1, the members of which are encompassed by the definition of the term “lignin-modifying enzyme”, as used herein.

Other lignin-modifying enzymes are encompassed by the Auxiliary Activity Family 2, as this family is defined in CAZy, which includes manganese peroxidase (EC 1.11.1.13), versatile peroxidase (EC 1.11.1.16), lignin peroxidase (EC 1.11.1.14), and peroxidase (EC 1.11.1.-).

In some embodiments, the lignin-modifying enzyme is a laccase enzyme variant derived or originating from a thermostable organism (a thermophilic organism). In some embodiments, the lignin-modifying enzyme is Tfu_1114, which is a laccase or a laccase-like enzyme naturally occurring in the bacterium Thermobifida fusca. T. fusca is an aerobic thermophilic soil bacterium with strong cellulolytic activity. This actinomycete produces seven different cellulases and has the ability to grow on xylan and it produces several enzymes involved in xylan degradation, such as xylanases, β-xylosidase, α-L arabinofuranosidase and acetylesterases.

In some embodiments, the LME is derived from species that include, without limitation, Thermobifida fusca, Citrobacter freundii B38, various E. coli species and Salmonella enterica subsp. diarizonae, Campylobacter coli, Campylobacter jejuni, Crinalium epipsammum, Deinococcus peraridilitoris, Desulfotomaculum gibsoniae, Melioribacter roseus and Thermacetogenium phaeum.

Carbohydrate Active Enzyme:

As used herein, the term “carbohydrate active enzyme” refers to an enzyme that catalyses the breakdown of carbohydrates and glycoconjugates. The term encompasses enzymatically active portions of enzymes that catalyse the breakdown of carbohydrates and glycoconjugates. The broad group of carbohydrate active enzymes is divided into enzyme classes and further into enzyme families according to a standard classification system [Cantarel et al., Nucleic Acids Res, 2009, 37, D233-238]. According to some embodiments of the present invention, the term “carbohydrate active enzyme” refer to any enzyme that belongs to any one of three classes of carbohydrates- and glycoconjugates-degrading enzymes, namely (i) glycoside hydrolases (GHs), which hydrolyze glycosidic bonds between two or more carbohydrates or between a carbohydrate and a non-carbohydrate moiety, including for example, cellulases, xylanase, α-L-arabinofuranosidase, cellobiohydrolase, β-glucosidase, β-xylosidase, β-mannosidase and mannanase; (ii) polysaccharide lyases (PLs), which catalyze the breakage of a carbon-oxygen bond in polysaccharides leading to an unsaturated product and the elimination of an alcohol, for example, pectate lyases and alginate lyases; and (ii) carbohydrate esterases (CEs), which catalyze the de-O or de-N-acylation of substituted saccharides, for example, acetylxylan esterases, pectin methyl esterases, pectin acetyl esterases and ferulic acid esterases. An informative and updated classification of carbohydrate active enzymes is available on the Carbohydrate-Active Enzymes (CAZy) server (www.cazy.org).

In some embodiments, the term “carbohydrate active enzyme” refers to any enzyme, which catalyses the degradation of carbohydrates that are found in cellulose and/or hemicellulose.

In some embodiments, the carbohydrate-active enzyme is a cellulosomal enzyme. In some embodiments, the carbohydrate-active enzyme is a non-cellulosomal enzyme.

Along with the classification system, a unifying scheme for designating the different catalytic modules and the different carbohydrate active enzymes was suggested and has been widely adopted. A catalytic module is designated by its enzyme class and family number. For example, a glycoside hydrolase having a catalytic module classified in family 10 is designated as “GH10”. An enzyme is designated by the type of activity, the family it belongs to and typically an additional letter. For example, a cellulase from a certain organism having a catalytic module classified as family 5 glycoside hydrolase (GH5), which is the first reported GH5 cellulase from this organism, is designated as “Cel5A”.

According to some embodiments of the present invention, the carbohydrate-active enzyme is a cellulase, a hemicellulose, a glycoside hydrolase, an exoglucanase, an endoglucanase, a xylanase, an exoxylanase, an endoxylanase, a mannanase, an arabinase, an arabinofuranosidase, a xyloglucanase, a β-xylosidase, a β-glucosidase, a polysaccharide lyase, a mannosidase and carbohydrate esterase.

According to some embodiments of the present invention, the carbohydrate-active enzyme is a cellulase and/or a hemicellulase classified in a glycoside hydrolase (GH) family selected from the group consisting of GH5, GH6, GH7, GH8, GH9, GH10, GH11, GH12, GH26, GH30, GH43, GH44, GH45, GH48, GH51, GH61, GH74, GH81, GH98 and GH124.

Exemplary endoglucanases, which are suitable for use as a carbohydrate-active enzyme in lignocellulolytic multi-enzyme complex presented herein include, without limitation, EC 3.2.1.4 of the GH5, GH6 EC 3.2.1.4, GH8 EC 3.2.1.4, GH9 EC 3.2.1.4, GH9 EC 3.2.1.74, GH16 EC 3.2.1.39, GH16 EC 3.2.1.6, GH44 EC 3.2.1.4, GH81 EC 3.2.1.39 and GH124 EC 3.2.1.4.

Exemplary exoglucanase, which are suitable for use as a carbohydrate-active enzyme in lignocellulolytic multi-enzyme complex presented herein include, without limitation, GH48 EC 3.2.1.91 and GH6 EC 3.2.1.176.

Exemplary endoxylanase, which are suitable for use as a carbohydrate-active enzyme in lignocellulolytic multi-enzyme complex presented herein include, without limitation, GH10 EC 3.2.1.8, GH11 EC 3.2.1.8, GH11EC 3.2.1.32, GH30 EC 3.2.1.8, GH43 EC 3.2.1.8, GH98 EC 3.2.1.8.

According to some embodiments of the present invention, the lignocellulolytic multi-enzyme complex presented herein includes at least one cellulase and at least one hemicellulase. Carbohydrate active enzymes that participate in the degradation of hemicelluloses, a heterogeneous group of branched and linear polysaccharides that are bound via hydrogen bonds to the cellulose microfibrils in the plant cell wall, are sometimes referred to as hemicellulases. Non-limiting examples of such carbohydrate active enzymes include cellulases, xylanases, mannanases, α-L-arabinofuranosidases, ferulic acid esterases, acetyl-xylanesterases, α-D-glucuronidases, β-xylosidases, 3-mannosidases, β-glucosidases, acetyl-mannanesterases, α-galactosidase, α-L-arabinanase and β-galactosidase.

The origin of the carbohydrate-active enzyme can be any microorganism (fungi or bacteria) that exhibit the ability to degrade cellulose and hemicellulose. In some embodiments, the carbohydrate-active enzymes are bacterial enzymes that are derived from species that include, without limitation, Thermobifida fusca, Clostridium thermocellum, Geobacillus stearothermophilus, Clostridium clariflavum, Clostridium (Ruminiclostridium) thermocellum, Clostridium cellulolyticum, Clostridium cellulovorans, Clostridium clariflavum, Clostridium papyrosolvens, Clostridium josui, Clostridium alkalicellulosi, Bacteroides (Pseudobacteroides) cellulosolvens, Acetivibrio cellulolyticus, Caldicellulosiruptor bescii (Anaerocellum thermophilum), Caldicellulosiruptor saccharolyticus DSM 890, Caldicellulosiruptor obsidiansis, Caldicellulosiruptor lactoaceticus, Caldicellulosiruptor kronotskyensis, Caldicellulosiruptor kristjanssonii, Ruminococcus albus, Ruminococcus bromii, Ruminococcus champanellensis and Ruminococcus flavefaciens. Thus, a carbohydrate-active enzyme can be a variant of the abovementioned enzymes derived from any of the abovementioned species and other species.

In some embodiments, the carbohydrate active enzymes include xylanases. Xylanases are classified, for example, in glycoside hydrolase families such as, but not limited to 5, 8, 10, 11, 26, 30, 43 and 74. In some embodiments, the xylanases are bacterial xylanases.

In some embodiments, the carbohydrate active enzymes include cellulases. The cellulases may be selected from exoglucanases, endoglucanases and proccessive-endoglucanase. Cellulases are classified, for example, in glycoside hydrolase families such as, but not limited to 5, 6, 7, 8, 9, 12, 26, 44, 45, 48, 51, 61, 74, and 124. In some embodiments, the cellulases are bacterial cellulases.

In some embodiments, the carbohydrate active enzymes include β-glucosidases. β-glucosidases are classified, for example, in glycoside hydrolase families such as, but not limited to 1, 3, 9, 30 and 116. In some embodiments, the β-glucosidases are bacterial β-glucosidases.

In some exemplary embodiments, a plurality of carbohydrate active enzymes bound to the scaffold polypeptide of the lignocellulolytic multi-enzyme complex presented herein include an exoglucanase, an endoglucanase, and a processive-endoglucanase.

In some embodiments, a lignocellulolytic multi-enzyme complex of the present invention includes more than one cellulase, at least two cellulases, for example three cellulases, four cellulases, or more. Each possibility represents a separate embodiment of the present invention.

In some embodiments, a lignocellulolytic multi-enzyme complex of the present invention includes more than one xylanase, at least two xylanases, for example xylanases cellulases, xylanases cellulases, or more. Each possibility represents a separate embodiment of the present invention.

In some specific exemplary embodiments, the plurality of carbohydrate active enzymes includes at least one of the exoglucanase Cel48S from C. thermocellum, the endoglucanase Cel8A from C. thermocellum and the proccessive-endoglucanases Cel9K and Cel9R from C. thermocellum.

In additional specific exemplary embodiments, the plurality of carbohydrate active enzymes includes at least one of the exoglucanase Cel48A from T. fusca, the endoglucanase Cel5A from T. fusca, and the proccessive-endoglucanase Cel9A from T. fusca.

In additional specific exemplary embodiments, the plurality of carbohydrate active enzymes includes at least one of the xylanases Xyn43A, Xyn11A, Xyn10B, and Xyn10A from T. fusca.

In some exemplary embodiments, a plurality of carbohydrate active enzymes bound to a scaffold polypeptide includes xylanases and an exoglucanase.

In some specific exemplary embodiments, the plurality of carbohydrate active enzymes includes the xylanases Xyn43A, Xyn11A, Xyn10B, and Xyn10A from T. fusca, and the exoglucanase Cel5A from T. fusca.

Exemplary enzymatic subunits with suitable dockerins are provided in the Examples section below.

Scaffold Polypeptide:

For some combinations of LMEs and CAEs, the arrangement, relative order and ratio within the complex has an effect on the overall activity. The effect of the arrangement of the activity of the complex can be readily determined by a person skilled in the art, and be altered and controlled by manipulation of the scaffold polypeptide component of the complex.

The components of the lignocellulolytic multi-enzyme complex presented herein are composed of a plurality of functional domains that interact with each other and with the lignocellulosic substrate. One of these components is a protein or a glycoprotein that comprises a distinctive non-catalytic scaffolding polypeptide that integrates the various enzyme components into one cohesive complex, by combining each of its “cohesin” domains with a corresponding “dockerin” domain present on each of the enzyme components, while the high-affinity cohesin-dockerin interaction defines the complex's structure. In some embodiments, the scaffold polypeptide further includes a substrate-binding module that adheres the substrate to the complex. The scaffold polypeptide also structurally organizes and sets the ratio between the various enzymatic components of the complex.

As used herein, the term “scaffold polypeptide”, “scaffold subunit” or “scaffoldin” are used interchangeably and refer to the assembly subunit that provides a plurality of binding sites for enzymatic and/or non-enzymatic protein components. Thus, the scaffold polypeptide serves as a platform for integration of components, both enzymes and non-enzymatic protein components. The scaffold polypeptide is typically non-catalytic. The scaffold polypeptide may include one or more substrate-binding modules.

In the context of embodiments of the present invention, the scaffold polypeptide may exhibit any number (n) of identical, similar or different dockerin modules (e.g., n=1-10) and any number (m) of identical, similar or different substrate-binding module substrate-binding modules (e.g., n=0-10).

Each section or domain in the scaffold polypeptide is linked to the neighbouring domain(s) via linkers or spacers, which are polypeptide chains of 1-100 amino acids. In some embodiments, the linkers are 1-10 amino acid long, 1-20, 1-30, 1-40, 1-50, 5-10, 5-20, 5-40, 5-30, 5-50, 10-20, 10-30, 10-40, 10-50, 20-30, 20-40, 20-50, 20-60, 50-60, 50-70, 50-80, 50-90 or 50-100 amino acids long. In some embodiments, linkers also connect between two or more domains in a catalytic component of the complex, as exemplified below for a chimeric enzyme component.

Cohesin and Dockerin Modules:

The assembly of the multi-enzyme complex according to embodiments of the present invention is mediated by a protein-protein interaction between two modules—cohesins and dockerins. In natural cellulosome complexes and on some artificial cellulosomes, cohesin and dockerin modules govern the integration of enzymes into a scaffoldin subunit, as well as the attachment of the cellulosome to the surface of a cellulosome-producing microorganism (in some cellulosome-producing microorganisms).

The cohesins are modules of approximately 140 amino acid residues long that typically appear as repeats as part of the structural scaffoldin subunit. There are three major types of cohesin modules, types I, II and III, which are classified based on amino acid sequence homology and protein topology. Classification of a given cohesin can be carried out through sequence alignment to known cohesin sequences. The sequence of type-II cohesin domains are characterized by two insertions which are not found in type-I cohesin domains. Topologically, all cohesin types share a common structure of nine-stranded β-sandwich with jellyroll topology. Type I cohesin includes only the basic jellyroll structure. The structure of the type-II cohesin module has an overall fold similar to that of type-I, but includes distinctive additions in the form of two “β-flaps” interrupting strands 4 and 8 and an α-helix at the crown of the protein module. The structure of the type-III cohesin module is similar to that of type-II, namely, it includes two β-flaps interrupting strands 4 and 8 and an α-helix, but the location of the α-helix differs from that of type-II. In addition, type-III is characterized by an extensive N-terminal loop.

The dockerins are modules of approximately 60-70 amino acid residues long, characterized by two duplicated 22-residue segments, frequently separated by a linker of 9-18 residues. The two repeats include a calcium-binding loop and an “F-helix” motif. The dockerins are classified into types according to the cohesin with which they interact, and similarly include types I, II and III. The phylogenetic map of the dockerins reflects to a great extent that of their cohesin counterparts, such that dockerins that interact with type-I cohesins are closely grouped, and the dockerins that interact with the type-II cohesins are also grouped and distant from the first group.

In general, the cohesin-dockerin interaction is highly specific, such that each cohesin binds to one dockerin and together this couple forms an affinity pair. In the context of embodiments of the present invention, a cohesin-dockerin affinity pair is said to have a matching pair members, and therefore the lignocellulolytic multi-enzyme complex presented herein is said to exhibit matching pairs of cohesin-dockerin. Thus, in some embodiments of the lignocellulolytic multi-enzyme complex presented herein, each of the LME and the CAE components exhibits a dockerin module that matches by specific affinity towards at least one of cohesin module in the scaffold polypeptide. In some embodiments of the present invention each of the enzymatic components of the complex exhibit a dockerin module that matches a single cohesin module in the scaffold polypeptide, thereby the sequential order and spatial proximity, as well as the ratio between the various enzymatic components of the complex are determined and controlled.

Interactions among type-I modules generally observe cross-species stringency of the cohesin-dockerin system, such that type-I cohesin of one microorganism species would not be expected to recognize type-I dockerins from a different microorganism species. Within a given species, however, type-I interactions tend to be non-specific, such that all cohesins on a primary scaffoldin tend to bind similarly to different enzyme-borne dockerins. Thus, within a given species, cohesin modules that serve for enzyme incorporation generally have similar specificities. Inter-species specificity of interactions among type-II modules appears to be less strict than that observed for type-I, and cross-species interaction is sometimes observed. There is essentially no cross-specificity between type I and type II cohesin-dockerin partners.

The cohesin modules constitute the scaffold polypeptide subunits, which are separated by 1-100 amino acid chains referred to herein as linkers or spacers. Dockerin modules with corresponding binding specificity are selected for the enzymes to be integrated into the complex. For the construction of a scaffold subunit that integrates enzymes to precise locations, cohesins of varied (divergent) specificities are be selected. For example, each cohesin can originate from a different microorganism. As another example, cohesins from the same species but of different types can be selected in order to achieve unique selectivity in the binding order and ratio of the various enzymatic components of the complex.

Information about classification of cohesin and dockerin modules can be found in the literature [e.g., Alber et al., Proteins, 2009, 77, 699-709; Noach et al., J. Mol. Biol., 2005, 348, 1-12; Xu et al., J. Bacteriol., 2003, 185, 4548-4557; Bayer et al., Annu. Rev. Microbiol., 2004, 58, 521-54; and Peer et al., FEMS Microbiol Lett., 2009, 291(1), 1-16]. Information about inter- and intra-species specificity among type I and type II cohesins and dockerins may be found in the literature [e.g., Haimovitz et al., Proteomics, 2008, 8, 968-979].

Non-limiting examples of cohesin-dockerin affinity pairs with mutual binding specificities that can be used for the construction of multi-enzyme complexes according to embodiments of the present invention are specified in Table 1 below, the sequences of which can be found in public databases and the literature [e.g., Barak, Y. et al., J Mol Recognit, 2005, 18, 491-501]:

TABLE 1

| Species of | Cohesin | Dockerin |
|---|---|---|
| Origin | Name | Name |
| C. thermocellum | cohesin of CipA (e.g., second | dockerin of Cel48S |
| | or third cohesin) | |
| | | dockerin of Xyn10Z |
| B. cellulosolvens | cohesin of ScaB (e.g., third | dockerin of ScaA |
| | cohesin) | |
| A. cellulolyticus | cohesin of ScaC (e.g., third | dockerin module of |
| | cohesin) | ScaB |
| C. thermocellum | type II cohesin module from a | type II dockerin |
| | cell surface-anchoring protein: | module from CipA |
| | Orf2p, SdbA, OlpB, | |
| | Cthe_0735 and Cthe_0736 | |
| C. cellulolyticum | cohesin from scaffoldin C (e.g., | dockerin from |
| | cohesin 1) | scaffoldin A |
| R. flavefaciens | cohesin from scaffoldin B of | dockerin from ScaA |
| | strain 17 (e.g., cohesin 1) | |
| A. fulgidus | cohesin 2375 | dockerin 2375 |

Examples of additional cohesin-dockerin pairs are available in the scientific literature and are known to persons of skill in the art.

Interacting cohesin and dockerin pairs can be taken from natural cellulosome-producing bacteria, for example, from scaffoldins and/or enzymes found in Acetivibrio cellulolyticus, Archaeoglobus fulgidus, Bacteroides cellulosolvens, Pseudobacteroides cellulosolvens, Clostidium alkalicellulosi, Clostridium acetobutylicum, Clostridium bornimense, Clostridium cellobioparum, Clostridium cellulolyticum, Clostridium cellulovorans, Clostridium clariflavum, Clostridium josui, Clostridium papyrosolvens, Clostridium perfringens, Clostridium saccharoperbutylacetonicum, Clostridium sp. BNL1100, Clostridium straminisolvens, Clostridium termitidis, Clostridium thermocellum, Ruminococcus albus, Ruminococcus bromii, Ruminococcus champanellensis and Ruminococcus flavefaciens.

Interacting cohesin and dockerin pairs can also be taken from non-cellulosomal bacteria and archaea [Bayer et al., FEBS Lett., 1999, 463, 277-280]. A non-limiting list of non-cellulosomal cohesin and dockerin modules can be found in the supporting information of Peer et al. [Peer et al., FEMS Microbiol Lett., 2009, 291, 1-16], which is incorporated herein by reference.

Substrate-Binding Module:

The complex provided herein may exhibit one or more substrate-binding modules (SBM), which assist in forming proximity between the complex and the lignocellulosic substrate. In the context of embodiments of the present invention, the term “substrate-binding module” refers to any molecular entity that can bind to the lignocellulosic substrate, and can contain one or more polypeptide chains, glycopeptides, polysaccharides, polynucleotides and combinations thereof. In some embodiments, the terms “substrate-binding module”, “cellulose-binding module” and carbohydrate-binding module” (CBM) are used interchangeably, while the same terms where the word “module” is replaced with the word “domain” (SBD or CBD), it is meant that the module is essentially or substantially a polypeptide. Carbohydrate-binding module (CBM) is a protein domain found in carbohydrate-active enzymes.

In some embodiments, the CBM or CBD is a contiguous amino acid sequence forming an integral part of the scaffold polypeptide (scaffoldin), or in some embodiments, the CBM or CBD is a contiguous amino acid sequence within a carbohydrate-active enzyme, exhibiting a discreet fold having carbohydrate-binding activity.

In some embodiments, the substrate-binding module is derived from naturally occurring scaffoldin sequences of any species, CAEs or any species, or other non-catalytic sugar or carbohydrate-binding protein of any species, such as lectins and sugar transport proteins. Exemplary microorganisms which can serve as sources for substrate-binding modules include, without limitation, clostridial and related genera (notably Ruminiclostridium species), including Clostridium (Ruminiclostridium) thermocellum, Clostridium cellulolyticum, Clostridium cellulovorans, Clostridium clariflavum, Clostridium papyrosolvens, Clostridium josui, Clostridium alkalicellulosi, Bacteroides (Pseudobacteroides) cellulosolvens, Acetivibrio cellulolyticus, Caldicellulosiruptor (Anaerocellum) species, Caldicellulosiruptor bescii (Anaerocellum thermophilum), Caldicellulosiruptor saccharolyticus DSM 890, Caldicellulosiruptor obsidiansis, Caldicellulosiruptor lactoaceticus, Caldicellulosiruptor kronotskyensis and Caldicellulosiruptor kristjanssonii.

Additional information regarding substrate-binding modules can be found in the literature, for example in U.S. Pat. Nos. 5,837,814, 5,496,934, 5,202,247 and 5,137,819, and in Khazanov, N. et al. [J. Phys. Chem. B, 2016, 120(2), pp 309-319]

A Chimeric Multifunctional Enzyme:

Any one of the enzymatic components of the complex presented herein can be in a form of multi-domain protein, having at least one catalytic domain and one dockerin module, and optionally at least one SBM.

In addition, any of the enzymatic component may exhibit an additional tag that endows additional functionality to the enzyme, such as, without limitation, a solubilization tag, an affinity binding tag, a detection tag, a fluorescence tag a chromatography tag, an epitope tag, a protein purification tag and a protein tag. Exemplary tags include, without limitation, AviTag, a peptide allowing biotinylation by the enzyme BirA and so the protein can be isolated by streptavidin; Calmodulin-tag, a peptide bound by the protein calmodulin; polyglutamate tag, a peptide binding efficiently to anion-exchange resin such as Mono-Q; E-tag, a peptide recognized by an antibody; FLAG-tag, a peptide recognized by an antibody; HA-tag, a peptide from hemagglutinin recognized by an antibody; His-tag, 5-10 histidines bound by a nickel or cobalt chelate; Myc-tag, a peptide derived from c-myc recognized by an antibody; NE-tag, an 18-amino-acid synthetic peptide recognized by a monoclonal IgG1 antibody; S-tag, a peptide derived from Ribonuclease A; SBP-tag, a peptide which binds to streptavidin; Softag 1, suitable for mammalian expression; Softag 3, suitable for prokaryotic expression; Strep-tag, a peptide which binds to streptavidin or the modified streptavidin called streptactin; TC tag, a tetracysteine tag that is recognized by FlAsH and ReAsH biarsenical compounds; V5 tag, a peptide recognized by an antibody; VSV-tag, a peptide recognized by an antibody; and Xpress tag. Other covalent peptide and protein tags are also contemplated within the scope of embodiments of the present invention.

In some embodiments, an enzymatic component may also exhibit two, three, four or more enzymatic domains, rendering it a multifunctional enzyme that can catalyse multiple varied reactions. In some embodiments, the multifunctional enzyme is an artificial chimeric enzyme, having catalytic domains that are not found as a single polypeptide chain in nature, and even originate from different species.

According an aspect of some embodiments of the present invention, there is provided a chimeric enzyme that includes at least one lignin-modifying enzyme, at least one carbohydrate-active enzyme and a dockerin module. Such a chimeric enzyme exhibits both the cellulose/hemicellulose-degrading activity and the lignin-degrading activity in one polypeptide chain, as well as the ability to be integrated into the lignocellulolytic multi-enzyme complex presented herein by virtue of its dockerin module. In some embodiments, the dockerin module in the chimeric enzyme links between two enzymatic domains of the chimeric enzyme via linkers of 1-100 amino acid long chains.

In some embodiments, the LME is laccase, such as, for non-limiting example, Tfu_1114 originating from Thermobifida fusca. In some embodiments, the CAE is xylanase, such as, for non-limiting example, XynT6 derived from Geobacillus stearothermophilus.

A chimeric enzyme exhibiting a laccase domain and a xylanase domain, linked to each other via a dockerin domain and further exhibiting a tag on the xylanase domain has been prepared and used successfully as an enzymatic component in an exemplary lignocellulolytic multi-enzyme complex, as demonstrated by the chimeric enzyme Xyn-c-Lac (SEQ ID No. 1) in the Examples section that follows below.

In some embodiments, the chimeric enzyme further exhibits a SBM, as this is defined hereinabove. A chimeric enzyme that exhibits a LME, a CAE and a SBM constitutes a molecular entity that confer total lignocellulosic degradation, which differ from the lignocellulolytic multi-enzyme complex by being a single polypeptide chain entity. A chimeric enzyme that exhibits a LME, a CAE, a SBM and a dockerin module constitute a single macromolecule that that may exhibit total lignocellulosic degradation independently, as well as the capacity of being integrated into a lignocellulolytic multi-enzyme complex, as presented herein.

Process of Preparing the Complex and its Components:

The various protein components and polypeptides comprising the lignocellulolytic multi-enzyme complex of the present invention may be synthesized by expressing a polynucleotide molecule encoding the polypeptide in a host cell, for example, a microorganism cell transformed with the nucleic acid molecule.

According to an aspect of some embodiments of the present invention, there is provided a genetically modified host cell, which has been transformed to include polynucleotides hat encode a plurality of components that form the complex presented herein. The present invention thus provides genetically modified cells capable of producing the lignocellulosic multi-enzyme complex of the present invention. These cells are capable of producing, and typically secreting, the different components of the complex. In some embodiments, the genetically modified cell is selected from a prokaryotic and eukaryotic cell. In some embodiments, the genetically modified cell is transformed to produce any one or more of the components, including the chimeric enzymes, alone or in any combination. Each possibility represents a separate embodiment of the invention.

According to an aspect of some embodiments of the present invention, there is provided a genetically modified host cell, which has been transformed to include polynucleotides hat encode the chimeric enzyme presented herein.

The synthesis of a polynucleotide encoding the desired polypeptide may be performed as described in the Examples below. DNA sequences encoding wild type polypeptides may be isolated from any strain or subtype of a microorganism producing them, using various methods well known in the art [see for example, Sambrook, et al., Molecular Cloning: A Laboratory Manual, Third Edition, Cold Spring Harbor, N.Y., 2001]. For example, a DNA encoding the wild-type polypeptide may be amplified from genomic DNA of the appropriate microorganism by polymerase chain reaction (PCR) using specific primers, constructed on the basis of the nucleotide sequence of the known wild type sequence. The genomic DNA may be extracted from the bacterial cell prior to the amplification using various methods known in the art [see for example, Marek, P. M. et al., “Cloning and expression in Escherichia coli of Clostridium thermocellum DNA encoding p-glucosidase activity”, Enzyme and Microbial Technology, 9(8), 1987, pp. 474-478]. The isolated polynucleotide encoding the wild type polypeptide may be cloned into a vector, such as the pET28a plasmid. An alternative method to producing a polynucleotide with a desired sequence is the use of a synthetic gene. A polynucleotide encoding a polypeptide of the present invention may be prepared synthetically, for example using the phosphoroamidite method [see, for example, Beaucage et al., Curr Protoc Nucleic Acid Chem, 2001, Chapter 3, Unit 3.3; and Caruthers et al., Methods Enzymol, 1987, 154:287-313]. The polynucleotide thus produced may then be subjected to further manipulations, including one or more of purification, annealing, ligation, amplification, digestion by restriction endonucleases and cloning into appropriate vectors. The polynucleotide may be ligated either initially into a cloning vector, or directly into an expression vector that is appropriate for its expression in a particular host cell type.

The polynucleotides may include non-coding sequences, including for example, non-coding 5′ and 3′ sequences, such as transcribed, non-translated sequences, termination signals, ribosome binding sites, sequences that stabilize mRNA, introns and polyadenylation signals. The polynucleotides may comprise coding sequences for additional amino acids heterologous to the variant polypeptide, in particular a marker sequence, such as a poly-His tag, that facilitates purification of the polypeptide in the form of a fusion protein.

Polypeptides may be produced as tagged proteins, for example to aid in extraction and purification. A non-limiting example of a tag construct is His-tag (six consecutive histidine residues), which can be isolated and purified by conventional methods. It may also be convenient to include a proteolytic cleavage site between the tag portion and the protein sequence of interest to allow removal of tags, such as a thrombin cleavage site.

The polynucleotide encoding the polypeptide may be incorporated into a wide variety of expression vectors, which may be transformed into in a wide variety of host cells. The host cell may be prokaryotic or eukaryotic.

Introduction of a polynucleotide into the host cell can be effected by well known methods, such as chemical transformation (e.g. calcium chloride treatment), electroporation, conjugation, transduction, calcium phosphate transfection, DEAE-dextran mediated transfection, transvection, microinjection, cationic lipid-mediated transfection, scrape loading, ballistic introduction and infection.

In some embodiments, the host cell is a prokaryotic cell. Representative, non-limiting examples of appropriate prokaryotic hosts include bacterial cells, such as cells of Escherichia coli and Bacillus subtilis. In other embodiments, the cell is a eukaryotic cell. In some exemplary embodiments, the cell is a fungal cell, such as yeast. Representative, non-limiting examples of appropriate yeast cells include Saccharomyces cerevisiae and Pichia pastoris. In additional exemplary embodiments, the cell is a plant cell.

The polypeptides may be expressed in any vector suitable for expression. The appropriate vector is determined according the selected host cell. Vectors for expressing proteins in E. coli, for example, include, but are not limited to, pET, pK233, pT7 and lambda pSKF. Other expression vector systems are based on beta-galactosidase (pEX); maltose binding protein (pMAL); and glutathione S-transferase (pGST).

Selection of a host cell transformed with the desired vector may be accomplished using standard selection protocols involving growth in a selection medium, which is toxic to non-transformed cells. For example, E. coli may be grown in a medium containing an antibiotic selection agent; cells transformed with the expression vector which further provides an antibiotic resistance gene, will grow in the selection medium.

Upon transformation of a suitable host cell, and propagation under conditions appropriate for protein expression, the desired polypeptide may be identified in cell extracts of the transformed cells. Transformed hosts expressing the polypeptide of interest may be identified by analyzing the proteins expressed by the host using SDS-PAGE and comparing the gel to an SDS-PAGE gel obtained from the host which was transformed with the same vector but not containing a nucleic acid sequence encoding the protein of interest.

The protein of interest can also be identified by other known methods such as immunoblot analysis using suitable antibodies, dot blotting of total cell extracts, limited proteolysis, mass spectrometry analysis, and combinations thereof.

The protein of interest may be isolated and purified by conventional methods, including ammonium sulfate or ethanol precipitation, acid extraction, salt fractionation, ion exchange chromatography, hydrophobic interaction chromatography, gel permeation chromatography, affinity chromatography, and combinations thereof.

The isolated protein of interest may be analyzed for its various properties, for example specific activity and thermal stability, using methods known in the art, some of them are described hereinbelow.

Conditions for carrying out the aforementioned procedures as well as other useful methods are readily determined by those of ordinary skill in the art (see for example, Current Protocols in Protein Science, 1995 John Wiley & Sons).

In particular embodiments, the polypeptides of the invention can be produced and/or used without their start codon (methionine or valine) and/or without their leader (signal) peptide to favor production and purification of recombinant polypeptides. It is known that cloning genes without sequences encoding leader peptides will restrict the polypeptides to the cytoplasm of the host cell and will facilitate their recovery (see for example, Glick, B. R. and Pasternak, J. J. (1998) In “Molecular biotechnology: Principles and applications of recombinant DNA”, 2nd edition, ASM Press, Washington D.C., p. 109-143).

Methods and Uses:

The present invention further provides compositions and systems that include the lignocellulosic multi-enzyme complex of the present invention, for use in biomass and lignocellulosic material degradation.

The present invention provides systems for bioconversion of cellulosic and/or lignocellulolytic material, the system comprising the lignocellulolytic multi-enzyme complex of the present invention.

The lignocellulolytic multi-enzyme complexes of the present invention, compositions comprising same, and cells producing same, may be utilized for the bioconversion of a cellulosic material into degradation products.

The terms “cellulosic materials”, “cellulosic biomass” and “lignocellulolytic material” refer to materials that contain cellulose, in particular materials derived from plant sources that contain cellulose. The cellulosic material encompasses lignocellulolytic material containing cellulose, hemicellulose and lignin. The lignocellulolytic material may include natural plant biomass and also paper waste and the like. Examples of suitable cellulosic and lignocellulolytic materials include, but are not limited to, wheat straw, switchgrass, corn cob, corn stover, sorghum straw, cotton straw, bagasse, energy cane, hard wood paper, soft wood paper, or combinations thereof.

Resulting sugars may be used for the production of alcohols such as ethanol, propanol, butanol and/or methanol, production of fuels, e.g., biofuels such as synthetic liquids or gases, such as syngas, and the production of other fermentation products, e.g. succinic acid, lactic acid, or acetic acid.

According to an aspect of the present invention, there is provided herein a method for converting cellulosic and/or lignocellulolytic material into degradation products, the method comprising exposing said cellulosic/lignocellulolytic material to the lignocellulolytic multi-enzyme complex of the present invention.

According to an additional aspect of the present invention, there is provided herein a method for converting cellulosic/lignocellulolytic material into degradation products, the method comprising exposing cellulosic/lignocellulolytic material to the lignocellulolytic multi-enzyme complex of the present invention, or to genetically modified cells capable of producing the lignocellulolytic multi-enzyme complex of the present invention.

The degradation products typically comprise mono-, di- and oligosaccharide, including but not limited to glucose, xylose, cellobiose, xylobiose, cellotriose, cellotetraose, arabinose, xylotriose.

Lignocellulolytic multi-enzyme complexes of the present invention may be added to bioconversion and other industrial processes, for example, continuously, in batches or by fed-batch methods. Alternatively or additionally, the lignocellulolytic multi-enzyme complexes of the invention may be recycled. By relieving end-product inhibition of endoxylanases and exo/endoglucanases (such as xylobiose and cellobiose), it may be possible to further enhance the hydrolysis of the cellulosic/lignocellulolytic material.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1—Materials and Methods

Cloning:

Recombinant laccases were cloned by using a two-step restriction free procedure [Unger, T. et al., J Struct Biol, 2010, 172, 34-44]. The plasmids and primers used for the restriction-free cloning are listed in Table 2. Other enzymes and scaffoldin were cloned using restrictions enzymes.

TABLE 2

| | | | Primary | Secondary |
|---|---|---|---|---|
| Vector | Forward primer | Reverse primer | PCR | PCR |
| name | (5′-3′) | (5′-3′) | template | template |
| pETduet-Lac | tataccatggtgacgggcac | ggtgctcgagtgggacc | T. fusca YX | pETduet |
| | cgt (SEQ ID No. 21) | ctccag (SEQ ID | genomic DNA | |
| | | No. 22) | | |
| pETduet-c-Lac | cggttcgccggatatgtctgg | ggtggcagcagcctag | pET21a- | pETduet- |
| | agggtcccaactagtcctgta | gttaattaagctgcttaag | Xy143-cl | Lac |
| | attgtat (SEQ ID No. | gtagcttacttacc | | |
| | 23) | (SEQ ID No. 24) | | |
| pETduet- | atgggcagcagccatcacc | accctggaagtacaggtt | pET9d-Xyn-c | pETduet-c- |
| Xyn-c-Lac | atcatcaccacaagaatgca | ttcacccgcggatttgtg | | Lac |
| | gattcctatgcgaaaaaacct | gtcgataatagcccaata | | |
| | (SEQ ID No. 25) | tgcggg (SEQ ID | | |
| | | No. 26) | | |
| pET28a | TATACCATGGcaca | gtcacctcggccgagtc | C. | pET28a |
| t48-A | tcaccatcaccatcacgcagt | gtggccgggtacctctgt | thermocellum | b-48A |
| | tgaaagcagttccac | aataa tgcggagtat | ATCC | |
| | (SEQ ID No. 27) | (SEQ ID No. 28) | genomic | |
| | | | DNA | |

Briefly, the plasmid pETduet-Lac was obtained as previously described [Chen, C-Y et al., Appl Microbiol Biotechnol, 2013, 97, pp. 8977-8986]. The pETduet-c-Lac plasmid was obtained by inserting a sequence coding for the dockerin module from Clostridium cellulolyticum Cel5A (termed “c”) [Moraïs, S. et al., mBio, 2011, 2, 1-11], amplified from the vector pET21a-Xy143-c [Moraïs, S. et al., mBio, 2012, 3, e00508-12-e00508-12], in pETDuet plasmid (Novagen). The Geobacillus stearothermophilus xylanase was obtained from a previous study [Barak, Y. et al., J Mol Recognit, 2005, 18, 491-501] and was inserted at the 5′ of the c-Lac coding sequence to produce the plasmid pETduet-Xyn-c-Lac. Similarly, t-48A chimaera (SEQ ID No. 4) was obtained by inserting a sequence coding for the dockerin module from Clostridium thermocellum XynZ (termed “t”), amplified from strain ATCC 27405 genomic DNA to replace the dockerin from Pseudobacteroides cellulosovens in pET28a b-48A [Caspi, J. et al., Applied and Environmental Microbiology, 2009, 75, pp. 7335-7342].

The Xyn11V-a (SEQ ID No. 3) chimera, was constructed by ligating sequentially the two modules into the linearized form of pET21a (Novagen Inc., Madison, Wis.). Xyn11V was first amplified using C. thermocellum ATCC 27405 genomic DNA using the primers 5′-ATTATGCATATGCACCATCACCATCACCACGATGTAGTAATTACGTCAA ACCAGAC-3′ (SEQ ID No. 18) and 5′-ATTCTACTCGAGATTATCACTAGTAGGTGTAGGTGTAGGATTTACA-3′ (SEQ ID No. 19) (NdeI, SpeI and XhoI sites in boldface) and inserted into pET21a linearized with NcoI and XhoI. Then, the dockerin from scaffoldin B was cloned from A. cellulolyticus genomic DNA using the primers 5′-CTACAACTAGTACTACAACACCAACGCCTAAAT-3′ (SEQ ID No. 20) and 5′-GGTGGTCTCGAGTTATTCTTCTTTCTCTTCAA-3′ (SEQ ID No. 8) (SpeI and XhoI sites in boldface) and inserted into pXyn11V linearized with SpeI and XhoI. Wild-type enzymes (XynT6, Cel5A and Ce148A) were cloned as described previously [Lapidot, A et al., J Biotechnol, 1996, 51, 259-264; Ghangas, G S. et al., Appl Environ Microbiol, 1987, 53,1470-5; and Irwin, D C. et al., Eur J Biochem, 2000, 267,4988-4997]. The plasmid encoding the chimeric glycoside hydrolases pET28a-f-5A was obtained from previous studies [Morais, S. et al., Appl Environ Microbiol, 2010, 76, 3787-3796]. All the enzyme constructs were designed to contain a His tag for subsequent purification.

The recombinant plasmid of scaffoldin ScafCA(CBM)TF (SEQ ID No. 5) was derived from pETscaf6 [Fierobe, H-P, et al., J Biol Chem, 2005, 280, 16325-16334]. pETscaf6, originally pET9d (Novagen Inc., Madison, Wis.) is composed of cohesin C (cohesin1 from scaffoldin C from C. cellulolyticum), CBM-T (CBM3a and cohesin 3 from the cellulosomal scaffoldin subunit C. thermocellum YS) and cohesin F (cohesin 1 from Ruminococcus flavefaciens strain 17 scaffoldin B). To construct ScafCA(CBM)TF (SEQ ID No. 5), cohesin A (cohesin 3 from A. cellulolyticus scaffoldin C) was amplified from the A. cellulolyticus genomic DNA using 5′-TATCGGGTACCGCGGCCGCATTTACAGGTTGACATTGGAAGT-3′ (SEQ ID No. 9) and 5′-TACGTGGTACCGATGCAATTACCTCAATTTT-3′ (SEQ ID No. 10) primers (KpnI and KpnI sites in boldface type) and ligated (T4 DNA ligase from Fermentas UAB, Vilnius, Lithuania) to pETscaf6-linearized using KpnI (all the restriction enzymes were purchased from New England Biolabs, Inc). Correct orientation of the cloned fragment was checked by restriction analysis of the resulting plasmid.

PCR were performed using Phusion High Fidelity DNA polymerase (New England Biolabs, Inc), PCR products were purified using a HiYield™ Gel/PCR Fragments Extraction Kit (Real Biotech Corporation, RBC, Taiwan) and plasmids were extracted using Qiagen miniprep kit (Qiagen, Netherlands). Plasmids were maintained and propagated in Escherichia coli DH5a.

Expression and Purification:

All recombinant proteins were expressed in E. coli BL21(DE3) stain, grown in autoinduction media [Studier, F W., Protein Expr Purif, 2005, 41, 207-234], and supplemented with the appropriate antibiotics. Protein purification was performed by immobilized metal-ion affinity chromatography on a Nickel-NTA column (Qiagen, Netherlands). ScafCA(CBM)TF (SEQ ID No. 5) was purified on phosphoric acid-swollen cellulose (PASC), 7.5 mg·ml-1 (pH 7), as previously described [Caspi, J. et al., Biocatal Biotransformation, 2006, 24, 3-12], followed by an additional purification step using Ni-NTA column.

Designer Cellulosome Assembly:

Designer cellulosome assembly was examined by both affinity pull down assay and by electrophoretic mobility in native and denaturing conditions as described earlier [Stern, J. et al., PLoS One, 2015, 10:e0127326].

Cohesin-Dockerin Interaction:

The procedure of Barak et al [Barak, Y. et al., J Mol Recognit, 2005, 18, 491-501] was followed to test the binding activity of the cohesin and dockerin components of the designer cellulosome.

Enzymatic Activity Assays:

The laccase activity assay was carried out in vertical shaker incubator at 50° C. for 15 minutes, using 1,5 v.1\4 enzyme and 2 mM substrate [Chen, C-Y. et al., Appl Microbiol Biotechnol, 2013, 97, 8977-8986]. Xylanase activity was assayed on xylan substrates by the DNS (dinitrosalicylic acid) method [Miller, G L., Anal Biochem, 1959, 31, 426-428]. Hatched wheat straw (blended at 0.2 to 0.8 mm, and containing 32% cellulose, 30% hemicellulose and 21% lignin [Morais, S. et al., mBio, 2012, 3, e00508-12-e00508-12]) was washed for 24 hours to remove residual reducing sugars. The enzymes and the scaffoldin at optimal molar ratios (determined in the Designer cellulosome assembly section), were incubated for 2 hours at 37° C. with 20 mM CaCl₂). Then, the complex or enzyme mixtures (at 0.5 μM, about 25 mg protein/g wheat straw for the largest complex) were assayed for wheat straw degradation in a 200 μl reaction (50 mM acetate buffer; pH 5.0, 12 mM CaCl₂), 2 mM EDTA, 7 g/l wheat straw). Reaction mixtures were incubated in a vertical shaker incubator for 72 hours at 50° C. All assays were performed in triplicates. To evaluate reducing sugars concentration, the DNS method was used [Miller, G L., Anal Biochem, 1959, 31, 426-428]. The 7-day activity assay was conducted similarly with 3.5 g/l of unpretreated wheat straw (instead of 7 g/l, about 49 mg protein/g wheat straw for the largest complex).

### Example 2—Results

Conversion of the Selected Enzymes to the Cellulosomal Mode:

In this study two T. fusca cellulases were used, the family 5 endoglucanase Cel5A and exoglucanase Ce148A, together with Clostridium thermocellum xylanase Xyn11V, which were integrated into a designer cellulosome. This combination of enzymatic activities was shown to be highly synergistic and efficient for the simultaneous hydrolysis of cellulose and hemicellulose. This complex was used as a base to evaluate the benefits of the incorporation in designer cellulosomes of a lignin-modifying enzyme, T. fusca laccase-like Tfu_1114 (hereafter termed “Lac”).

In order to convert T. fusca enzymes to the cellulosomal mode, dockerin modules originating from different bacterial species and showing different binding specificities were used. T. fusca Ce148A and Cel5A were fused at their N-termini with dockerins originated from Clostridium thermocellum and Ruminococcus flavefaciens, and termed “t-48A” (SEQ ID No. 4) and “f-5A” (SEQ ID No. 2) respectively [Morais, S. et al., Appl Environ Microbiol, 2010, 76, 3787-3796]. In both cases, the original N-terminal CBM2 was replaced by the dockerin module. C. thermocellum xylanase Xyn11V was fused to an Acetivibrio cellulolyticus dockerin (termed “a”). This enzyme displays the same modular arrangement as T. fusca Xyn11A, a GH11 module and a CBM2 (specific for xylan-binding and cellulose to lesser extent) [Fernandes, A C. et al., Biochem J, 1999, 342, 105-110] but is advantageous over the latter, since the recombinant form is expressed very efficiently in E. coli expression systems. Similar to the chimaeric form of T. fusca Xyn11A, the CBM was maintained in C. thermocellum Xyn11V to preserve enzymatic activity in designer cellulosomes as reported previously [Moraïs, S. et al., Appl Environ Microbiol, 2010, 76, 3787-3796], and the dockerin module was added at the C-terminus of the protein. The enzymatic activity of the resultant Xyn11V-a (SEQ ID No. 3) was comparable to the wild-type Xyn11V on xylan substrates (see, Table 3 below, presenting specific activities of the recombinant enzymes on various xylan substrates in terms of Katal/mol enzyme).

TABLE 3

| Substrate | Xyn11V | Xyn11V-a (SEQ ID No. 3) |
|---|---|---|
| Birch wood xylan | 619 ± 13 | 490 ± 34 |
| Oat spelt xylan | 658 ± 1 | 921 ± 19 |
| Beechwood xylan | 977 ± 8 | 1300 ± 20 |

The conversion of the laccase Tfu_1114 to the cellulosomal mode proved more difficult, since the chimaeric enzyme bearing the dockerin from Clostridium cellulolyticum at either the N- or C-terminus, failed to express in E. coli cells under various growth conditions. In order to overcome this barrier, a highly expressed GH10 xylanase XynT6, from the thermophile Geobacillus stearothermophilus, was used as a solubility tag [Barak, Y. et al., J Mol Recognit, 2005, 18, 491-501; and Lapidot, A. et al., J Biotechnol, 1996, 51, 259-264] and fused at the N-terminus of the dockerin-bearing laccase. The resulting bi-functional chimera was successfully expressed and purified. The XynT6 solubility tag was not removed subsequently and kept fused to the converted Tfu_1114, as its thermophilic xylanase activity is expected to further promote xylan degradation within the framework of our experiments. As a control, a variant of XynT6 fused only to the dockerin (termed “c”) was also produced, and termed “Xyn-c”.

Construction of the Tetravalent Scaffoldin:

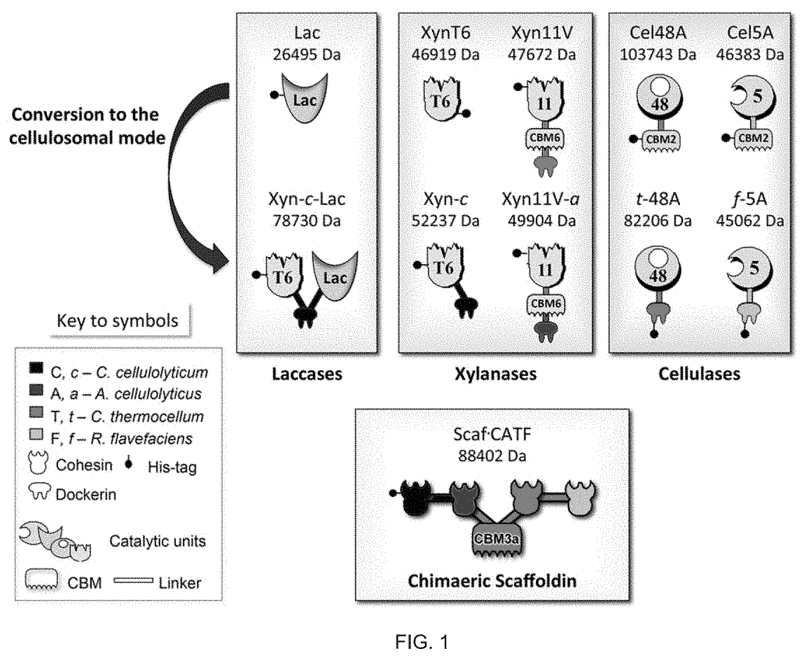

In order to drive the assembly of the above-described dockerin-bearing enzymes into a defined designer cellulosome, a tetravalent scaffoldin, ScafCA(CBM)TF (SEQ ID No. 5), containing the appropriate matching cohesins was produced. It includes four different cohesin types, originating from C. cellulolyticum (C), A. cellulolyticus (A), C. thermocellum (T), and R. flavefaciens (F). In order to target the scaffoldin to the cellulose-containing substrate, a cellulose-binding CBM3a from C. thermocellum was incorporated between A and T, as shown in FIG. 1.

FIG. 1 presents a schematic illustration of the recombinant proteins used in the example presented herein, wherein the numbers 5, 11, and 48 refer to the corresponding GH family (GH5, GH48, GH11) of the catalytic module; uppercase characters (A, C, F, T) indicate the source of the cohesin module and lowercase characters (a, c, f, t) indicate the source of the dockerin module.

Functionality of the Chimeric Bifunctional Enzyme Xyn-c-Lac:

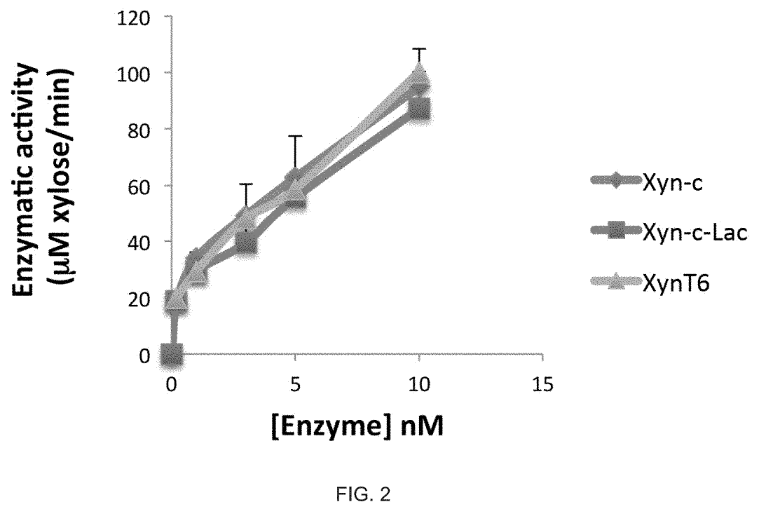

In order to assess whether the laccase and xylanase components of Xyn-c-Lac (SEQ ID No. 1) were affected by the translational fusion, enzymatic activities of the chimera were compared to the corresponding wild-type moieties. Xylanase activity was assessed by dinitrosalicylic acid (DNS) assay using beechwood xylan as a substrate and using previously reported reactions parameter for the wild-type xylanase XynT6 [Khasin, A. et al., Appl Environ Microbiol, 1993, 59, 1725-1730]. The xylanase moiety retained enzymatic activity and its activity profile was similar to the wild-type and Xyn-c-xylanases, as can be seen in FIG. 2.

FIG. 2 presents comparative plot of xylanase activity of XynT6, Xyn-c and Xyn-c-Lac (SEQ ID No. 1) on beechwood xylan at 50° C., wherein the assay was repeated twice and the error bars indicate the standard deviation from the mean of triplicate samples from one experiment.

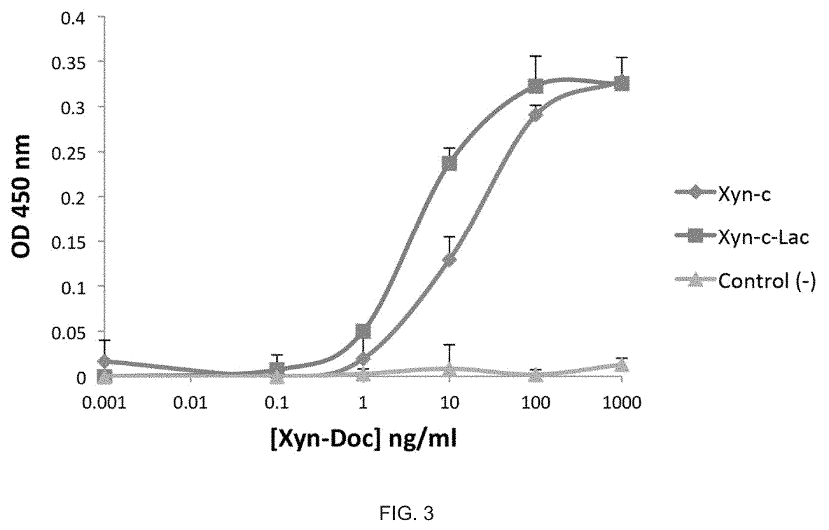

The functionality of the dockerin module was confirmed by affinity-based ELISA that measured the binding between the chimaeric of Xyn-c-Lac (SEQ ID No. 1) and the cohesin partner. The dockerin-bearing enzyme specifically bound the respective cohesin in an exclusive manner, as can be seen in FIG. 3.

FIG. 3 presents ELISA assay results obtained for cohesin-dockerin binding, wherein Xyn-c and Xyn-c-Lac (SEQ ID No. 1) interacted with the cohesin 1 from C. cellulolyticum CipC, and wherein Xyn-c-Lac (SEQ ID No. 1) failed to interact with C. thermocellum cohesin 3 from CipA as a negative control (error bars indicate the standard deviation from the mean of triplicate samples from one experiment).

In order to ensure that the addition of xylanase and dockerin does not affect the laccase activity in the chimaeric Xyn-c-Lac (SEQ ID No. 1), its activity was assayed and compared to that of the wild-type laccase on a variety of laccase substrates. In the first characterization of the wild-type laccase [Chen, C-Y et al., Appl Microbiol Biotechnol, 2013, 97, pp. 8977-8986], enzymatic assays were conducted with the 2,6 DMP substrate within a pH range of 6.5-9.0. The activity for oxidation was assayed by 15 minutes incubation of the enzymes at 50° C. with 20 mM of the substrates ABTS, 2,6-DMP, guaiacol and veratryl alcohol in various buffers and pH conditions.

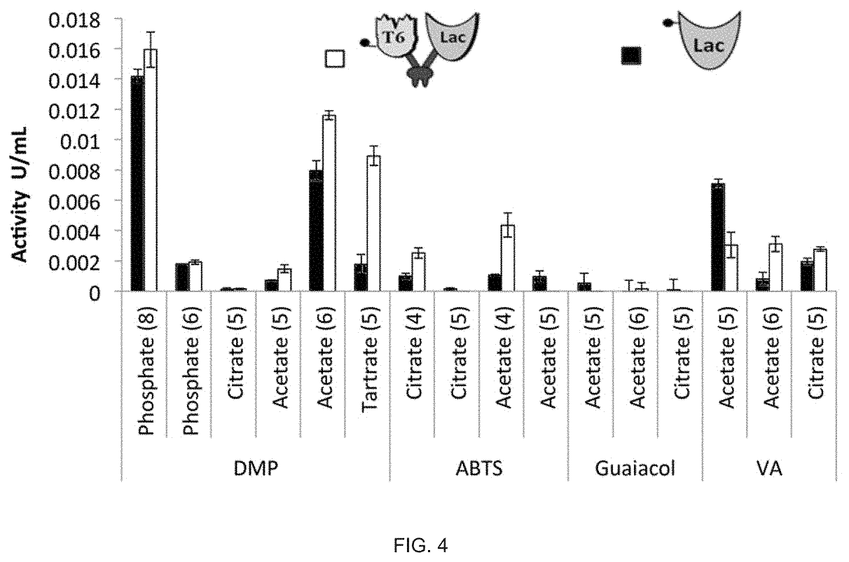

FIG. 4 presents comparative activity plot of the wild-type laccase (Lac) and chimeric Xyn-c-Lac (SEQ ID No. 1) towards different substrates.

The enzymatic activity of laccase towards 2,6-dimethoxyphenol (2,6-DMP; Syringol), 2,2′-azino-bis(3-ethylbenzothiazoline-6-sulphonic acid) (ABTS), guaiacol and veratryl alcohol (VA) was performed with different buffers in various pH conditions (pH value is given in parenthesis under the each bar pair). The oxidation rate was calculated based on the extinction coefficient of the oxidized product of each substrate. Each reaction was performed three times. Error bars represent standard deviations.

As can be seen in FIG. 4, the addition of the xylanase and dockerin moieties to the chimeric Xyn-c-Lac (SEQ ID No. 1) did not affect its activity in comparison to the wild-type enzyme under most of the conditions examined. Both the chimaeric enzyme and the wild-type enzyme exhibited the highest activity toward 2,6-DMP using phosphate buffer at pH 8. This result is in agreement with a previous study that characterized the wild-type enzyme activity. The substrate 2,6-DMP was oxidized by both the wild-type and the chimaeric enzymes in tartrate buffer (pH 5) and citrate buffer (pH 6). The second-highest oxidized substrate was veratryl alcohol (VA). Surprisingly, the chimaeric enzyme exhibited moderate activity for ABTS at pH 4, whereas the activity of the wild-type enzyme was very low. Only minor activity of both the wild-type and the chimaeric enzymes toward guaiacol were observed. Since the optimal pH of the laccase varied with substrate and buffering agent and the T. fusca enzymes cellulases and xylanases in the designer cellulosome machinery are known to be active at pH ranges of 5-6, pH 5 was elected as the working pH in the present experiments.

Analysis of Designer Cellulosome Complex Formation:

In order to examine the incorporation of the various components into the designer cellulosome complex, the ability of the complex to bind microcrystalline cellulose by virtue of its resident CBM was used to perform an affinity pull-down assay. The designer cellulosome complex was first incubated with cellobiose prior to its incubation with cellulose, in order to prevent the non-specific binding of the catalytic modules to cellulose. The bound and unbound fractions were examined by SDS-PAGE. Dockerin-bearing enzymes that failed to interact properly with the matching cohesin on the scaffoldin would appear in the unbound fraction. The four enzymes were incorporated together at their optimal ratio into the scaffoldin.

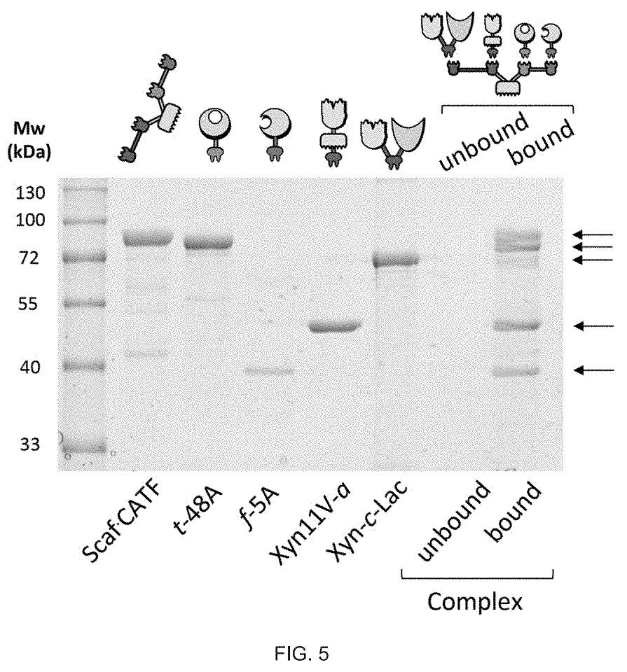

FIG. 5 presents an SDS-PAGE gel slab of the bound and unbound fractions of the various designer cellulosome preparations, according to some embodiments of the present invention, showing the results of the affinity pull-down assay serving for the assessment of cellulosome complex formation, wherein dockerin-bearing enzymes interacting properly with matching cohesins of the scaffoldin protein appear as bands in the bound fraction (marked with arrows), and no visible bands in the unbound fraction indicate enzymes that failed to interact properly with the matching cohesins of the scaffoldin.

As seen in FIG. 5, complex formation seems to be complete as all five proteins appear in the bound fraction, whereas the amount of protein observed in the unbound fraction is negligible. In addition, the assembly of the complex and its components was further confirmed by non-denaturing PAGE electrophoresis.

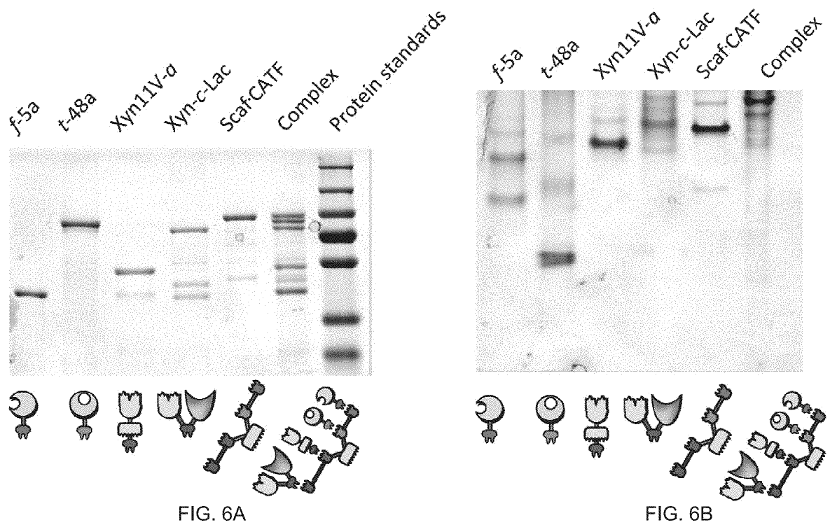

FIGS. 6A-B present the results of an electrophoresis mobility experiment to verify the formation of a designer cellulosome complex, according to some embodiments of the present invention, wherein FIG. 6A is an SDS-PAGE gel slab and FIG. 6B is a non-denaturing gel slab.

As can be seen in FIGS. 6A-B, electrophoretic mobility analysis of components and assembled complexes on non-denaturing and denaturing gels, using equimolar concentrations of the chimeric enzymes and their matching scaffoldin, indicates their near-complete interaction as a single major band formed (see, FIG. 6B).

Degradation of Untreated Wheat Straw:

In order to compare substrate degradation into reducing sugars, various free enzymes and scaffoldin complexes were used at 0.5 μM with a substrate concentration of 7 g/l.

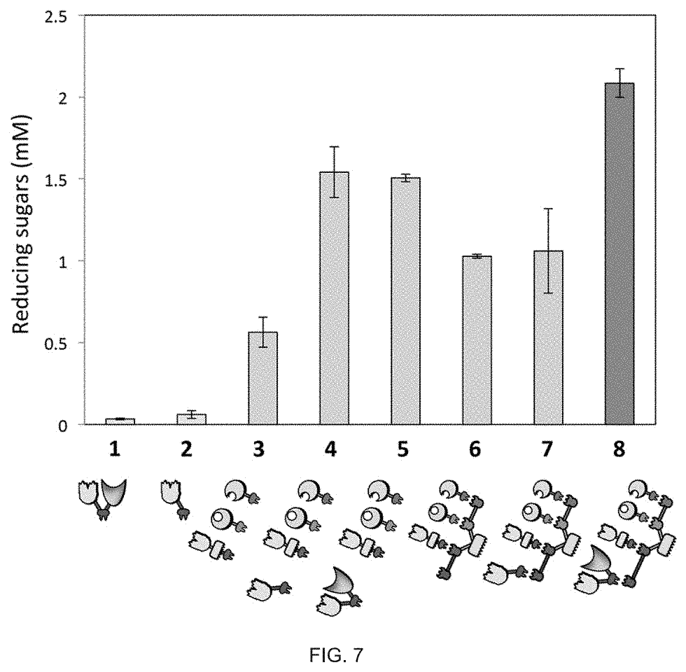

FIG. 7 presents a bar plot showing the comparative degradation of non-treated wheat straw incubated for 72 hours at 50° C. with laccase in tetravalent designer cellulosomes and free-enzyme combinations, wherein bar 1 represents substrate degradation by the bifunctional chimaeric xylanase-tagged laccase (Xyn-c-Lac (SEQ ID No. 1)), bar 2 represents substrate degradation by the xylanase tag alone (Xyn-c), bar 3 represents substrate degradation by the three free dockerin-bearing GHs (Xyn11V-a (SEQ ID No. 3), t-48A (SEQ ID No. 4) and f-5A (SEQ ID No. 2)) alone, bar 4 represents substrate degradation by the three free GHs with the additional xylanase tag (Xyn-c), bar 5 represents substrate degradation by the three free GHs with the addition of the xylanase-tagged laccase (Xyn-c-Lac (SEQ ID No. 1)), bar 6 represents substrate degradation by the three scaffoldin-complexed GHs, bar 7 represents substrate degradation by the scaffoldin-complexed GHs+xylanase tag, and bar 8 represents substrate degradation by the scaffoldin-complexed GHs+laccase, whereby each reaction was performed three times and error bars represent standard deviations.

As can be seen in FIG. 7, the reaction yields from bar 1 to bar 8 were 0.14%, 0.3%, 2.4%, 6.7%, 6.5%, 4.5%, 4.6% and 9% respectively, using the predetermined maximum sugar release of 3.3 mmoles reducing sugars/g dry matter [Ravachol, J. et al., Biotechnol Biofuels, 2015, 8,114, doi: 10.1186/s13068-015-0301-4]. As can further be seen in FIG. 7, the Xyn-c-Lac (SEQ ID No. 1) (bar 1) and the Xyn-c (bar 2) produced negligible amounts of reducing sugars after incubation with non-treated wheat straw. The combination of the three enzymes f-5A (SEQ ID No. 2), t-48A (SEQ ID No. 4) and Xyn11V-a (SEQ ID No. 3) into a designer cellulosome (bar 6) resulted in a 1.8 fold increase in enzymatic activity as compared to the mixtures of the free enzymes (bar 3). Xyn-c-Lac (SEQ ID No. 1) (bar 5) served to enhance the amount of reducing sugars by 2.7 fold when combined with the free enzymes (bar 3). This activity could in fact be attributed to the xylanase solubility tag (Xyn), since similar levels of reducing sugars were obtained while adding Xyn-c to the mixture of free enzymes (bar 4). In contrast, the incorporation of the Xyn-c-Lac (SEQ ID No. 1) into the designer cellulosome machinery (bar 8) generated an increase of 1.4 fold in the amount of reducing sugar production as compared to the mixture of the chimeric enzymes in their free state (bar 4), and a 2-fold increase as compared to the trivalent designer cellulosomes lacking the laccase (with or without the control Xyn-c) (bar 6 and bar 7) following 72 hours incubation with non-treated wheat straw. The fact that the combination of Xyn-c with the designer cellulosomal system (bar 7) did not serve to increase the amount of reducing sugar production demonstrates that the increase in activity caused by addition of Xyn-c-Lac (SEQ ID No. 1) to the designer cellulosome machinery (bar 8) can be attributed to the laccase moiety. Interestingly the Xyn-c enzyme contributed to the enzymatic activity in combination with the other enzymes in the free state only; as part of the designer cellulosome (bar 7), Xyn-c reduced the activity as compared to the free enzyme mixture (bar 4).

The results suggest a strong proximity effect between the laccase and the other enzymatic (glycoside hydrolase) partners and the necessity of the laccase to be located in the cellulosome complex to achieve efficient degradation.

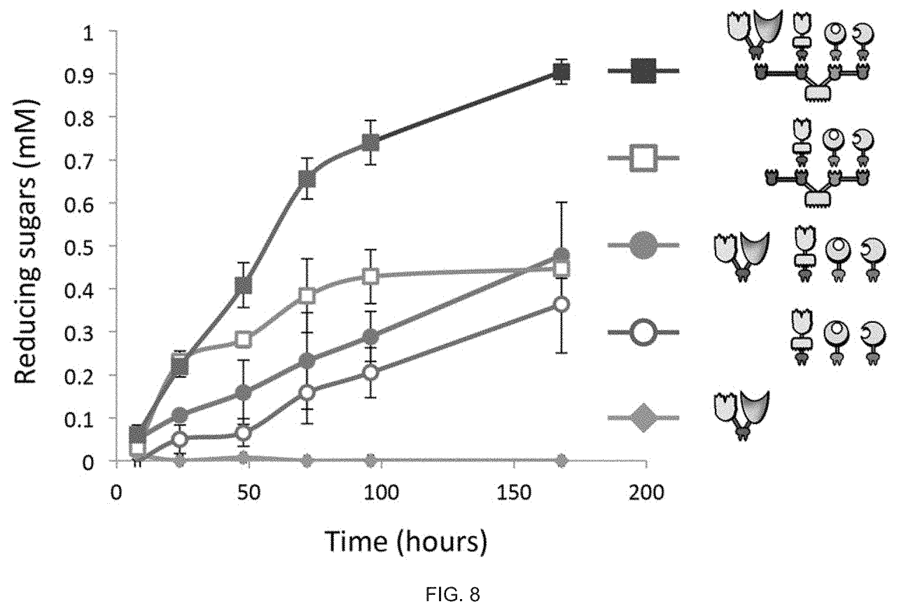

FIG. 8 presents kinetics studies over 7 days of the tetravalent designer cellulosome, according to some embodiments of the present invention, bearing the Xyn-c-Lac (SEQ ID No. 1) enzyme as compared to selected controls.

As can be seen in FIG. 8, after 7 days of incubation, the tetravalent designer cellulosome effected a 50% increase in the amount of reducing sugars in comparison to the trivalent designer cellulosome lacking the LME laccase. Together, these results demonstrate the ability of laccase to enhance cellulolytic and hemicellulolytic degradation when integrated into the designer cellulosome machinery.

### Example 3—Additional Exemplary Embodiment

The enzymatic activity of two exemplary designer cellulosomes complexes, according to some embodiments of the present invention, containing three scaffoldins and either four or five enzymes, were prepared and assayed on two agrofood waste products, brewer's spent grain and apple pomace.

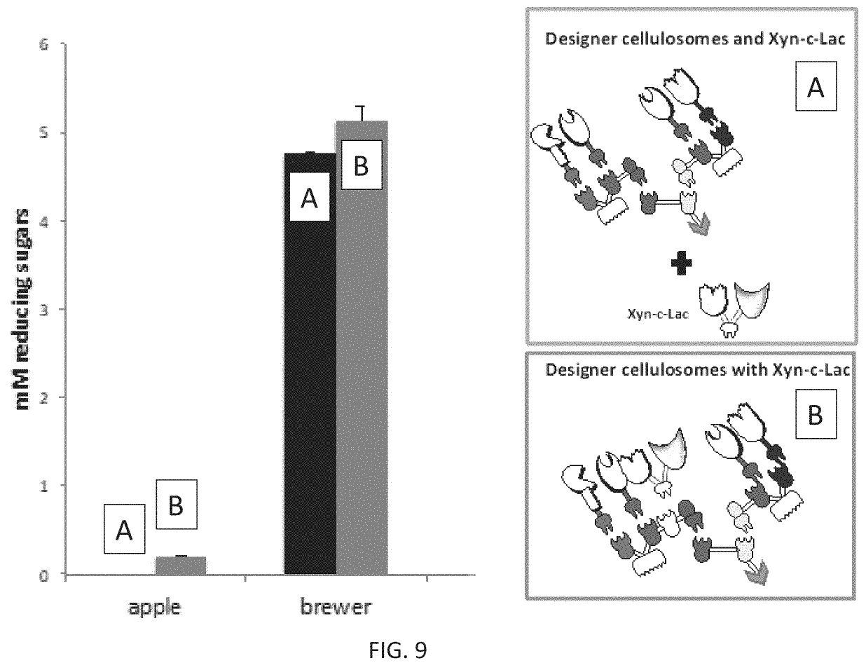

Enzyme activity was measured on 2% biomass at 30° C. and pH 5 after 72 hours of incubation period, and the results are presented in FIG. 9. Enzymatic activity is defined as mM soluble reducing sugars following the 72 hours reaction period. Each reaction was performed in triplicate, and standard deviations are indicated.

FIG. 9 presents a plot of comparative enzymatic activity of designer cellulosomes with an addition of the free Xyn-c-Lac (marked “A”), or containing the Xyn-c-Lac, according to some embodiments of the present invention (marked “B”) on degradation of brewer's spent grain and apple pomace.

The four enzymes containing designer cellulosome (marked “A” in FIG. 9) was composed of three cellulases (from C. papyrosolvens GH5, GH9 and T. fusca Cel6A), and one xylanase (T. fusca Xyn 11A), and supplemented with the bifunctional xylanase-laccase Xyn-c-Lac as free enzyme. The five enzymes designer cellulosomes (marked “B” in FIG. 9) had a similar composition but contained also the bifunctional xylanase-laccase Xyn-c-Lac (one of the scaffoldin was enlarged to contain an additional cohesin for its incorporation).

As can be seen in FIG. 9, designer cellulosomes containing the Xylanase-Laccase (Xyn-c-Lac) appeared more efficient than designer cellulosomes with the addition of the free Xylanase-Laccase (Xyn-c-Lac) on both types of substrates.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A lignocellulolytic multi-enzyme complex comprising at least one lignin-modifying enzyme and at least one carbohydrate-active enzyme, wherein said lignin-modifying enzyme is selected from the group consisting of a laccase (EC 1.10.3.2), a lignin peroxidase (EC 1.11.1.14), a manganese peroxidase (EC 1.11.1.13) and a versatile peroxidase (EC 1.11.1.16).

2. The complex of claim 1, further comprising a scaffold polypeptide, said scaffold polypeptide comprises at least one cohesin module, wherein said cohesin modules are separated by linkers that comprise 1-100 amino acids, and each of said lignin-modifying enzyme and said carbohydrate-active enzyme is having a dockerin module that matches at least one of said cohesin modules, said dockerin module is bound to said cohesin module.

3. The complex of claim 2, wherein said lignin-modifying enzyme is in a form of a chimeric enzyme that comprises said lignin-modifying enzyme and a carbohydrate-active enzyme, each attached to said dockerin module via a linker that comprises 1-100 amino acids.

4. The complex of claim 2, wherein each of said dockerin modules matches a single cohesin module in said scaffold polypeptide.

5. The complex of claim 2, wherein said scaffold polypeptide further comprises at least one substrate-binding module attached to at least one of said cohesin modules via linkers that comprise 1-100 amino acids.

6-7. (canceled)

8. The complex of claim 1, wherein said lignin-modifying enzyme is a laccase.

9. (canceled)

10. The complex of claim 1, wherein said carbohydrate-active enzyme is a cellulose- and/or hemicellulose-degrading enzyme.

11-18. (canceled)

19. A chimeric enzyme comprising a lignin-modifying enzyme, a carbohydrate-active enzyme and a dockerin module, wherein said lignin-modifying enzyme is selected from the group consisting of a laccase (EC 1.10.3.2), a lignin peroxidase (EC 1.11.1.14), a manganese peroxidase (EC 1.11.1.13) and a versatile peroxidase (EC 1.11.1.16).

20. The chimeric enzyme of claim 19, wherein each of said lignin-modifying enzyme and said carbohydrate-active enzyme is attached to said dockerin module via a linker that comprises 1-100 amino acids.

21. (canceled)

22. The chimeric enzyme of claim 19, wherein said lignin-modifying enzyme is a laccase.

23. (canceled)

24. The chimeric enzyme of claim 19, wherein said carbohydrate-active enzyme is selected from the group consisting of a cellulase, a hemicellulose, a glycoside hydrolase, an exoglucanase, an endoglucanase, a xylanase, an exoxylanase, an endoxylanase, a mannanase, an arabinase, an arabinofuranosidase, a xyloglucanase, a β-xylosidase, a β-glucosidase, a polysaccharide lyase, a mannosidase and carbohydrate esterase.

25. The chimeric enzyme of claim 24, wherein said carbohydrate-active enzyme is a cellulase classified in a glycoside hydrolase (GH) family selected from the group consisting of GH5, GH6, GH7, GH8, GH9, GH10, GH11, GH12, GH26, GH30, GH43, GH44, GH45, GH48, GH51, GH61, GH74, GH81, GH98 and GH124.

26. The chimeric enzyme of claim 25, wherein said carbohydrate-active enzyme is a xylanase.

27. (canceled)

28. The chimeric enzyme of claim 19, further comprising a tag selected from the group consisting of a solubilisation tag, an affinity binding tag, a detection tag, a fluorescence tag a chromatography tag, an epitope tag, a protein purification tag and a protein tag.

29. The chimeric enzyme of claim 19, having SEQ ID No. 1.

30. The chimeric enzyme of claim 19, further comprising at least one substrate-binding module.

31. The chimeric enzyme of claim 30, wherein said substrate-binding module is a cellulose-binding module.

32. The chimeric enzyme of claim 31, wherein said cellulose-binding module is a cellulose-binding domain (CBD).

33. A composition for degrading a cellulosic or lignocellulosic material comprising the complex of claim 1.

34. (canceled)

35. A method for degrading a cellulosic or lignocellulosic material, the method comprising exposing the cellulosic or lignocellulosic material to the complex of claim 1.

36-39. (canceled)


SEQUENCE LISTING (28 sequences)

SEQ ID NO: 1 - protein, 713 aa, Artificial sequence
  recombinant enzyme
MGSSHHHHHHKNADSYAKKPHISALNAPQLDQRYKNEFTIGAAVEPYQLQNEKDVQMLKRHFNSIVAENVMKPISIQPEEGKFNFEQADRIVKFAKANGMDIRFHTLVWHSQVPQWFFLDKEGKPMVNETDPVKREQNKQLLLKRLETHIKTIVERYKDDIKYWDVVNEVVGDDGKLRNSPWYQIAGIDYIKVAFQAARKYGGDNIKLYMNDYNTEVEPKRTALYNLVKQLKEEGVPIDGIGHQSHIQIGWPSEAEIEKTINMFAALGLDNQITELDVSMYGWPPRAYPTYDAIPKQKFLDQAARYDRLFKLYEKLSDKISNVTFWGIADNHTWLDSRADVYYDANGNVVVDPNAPYAKVEKGKGKDAPFVFGPDYKVKPAYWAIIDHKSAGENLYFQGTSPVIVYGDYNNDGNVDALDFAGLKKYIMAADHAYVKNLDVNLDNEVNAFDLAILKKYLLGMVSKLPSQDPNSVTGTVVELAPGIHAGFTGRAGGVSGEPYATLNLGDHVGDDPAAVAENRRRAALGFGISPDRVVWMNQVHGATAVTVTGSGQAGDVDAVVTPEAGLALAVLVADCLPLLVADAAAGVIGAAHAGRPGMAAGVVPALVAEMARHGARPERCVALLGPAICGRCYEVPRDLQDRVARTVPEARCTTAEGTPGLDIRAGVTAQLTNLGVTNITHDSRCTRESADLFSYRRDATTGRFAGYVWRVP

SEQ ID NO: 2 - protein, 411 aa, Artificial sequence
  recombinant enzyme
MAHHHHHHAPSPGTKLVPTWGDTNCDGVVNVADVVVLNRFLNDPTYSNITDQGKVNADVVDPQDKSGAAVDPAGVKLTVADSEAILKAIVELITLPQAVPGTQPGTGTPVERYGKVQVCGTQLCDEHGNPVQLRGMSTHGIQWFDHCLTDSSLDALAYDWKADIIRLSMYIQEDGYETNPRGFTDRMHQLIDMATARGLYVIVDWHILTPGDPHYNLDRAKTFFAEIAQRHASKTNVLYEIANEPNGVSWASIKSYAEEVIPVIRQRDPDSVIIVGTRGWSSLGVSEGSGPAEIAANPVNASNIMYAFHFYAASHRDNYLNALREASELFPVFVTEFGTETYTGDGANDFQMADRYIDLMAERKIGWTKWNYSDDFRSGAVFQPGTCASGGPWSGSSLKASGQWVRSKLQS

SEQ ID NO: 3 - protein, 454 aa, Artificial sequence
  recombinant enzyme
MHHHHHHDVVITSNQTGTHGGYNFEYWKDTGNGTMVLKDGGAFSCEWSNINNILFRKGFKYDETKTHDQLGYITVTYSCNYQPNGNSYLGVYGWTSNPLVEYYIIESWGTWRPPGATPKGTITVDGGTYEIYETTRVNQPSIKGTATFQQYWSVRTSKRTSGTISVTEHFKAWERLGMKMGKMYEVALVVEGYQSSGKADVTSMTITVGNAPSTSSPPGPTPEPTPRSAFSKIEAEEYNSLKSSTIQTIGTSDGGSGIGYIESGDYLVFNKINFGNGANSFKARVASGADTPTNIQLRLGSPTGTLIGTLTVASTGGWNNYEEKSCSITNTTGQHDLYLVFSGPVNIDYFIFDSKGVNPTPTPTSTTTPTPKFIYGDVDGNGSVRINDAVLIRDYVLGKINEFPYEYGMLAADVDGNGSIKINDAVLVRDYVLGKIFLFPVEEKEELEHHHHHH

SEQ ID NO: 4 - protein, 739 aa, Artificial sequence
  recombinant enzyme
MAHHHHHHAVESSSTGLGDLNGDGNINSSDLQALKRHLLGISPLTGEALLRADVNRSGKVDSTDYSVLKRYILRIITEVPGHDSAEVTVREIDPNTSSYDQAFLEQYEKIKDPASGYFREFNGLLVPYHSVETMIVEAPDHGHQTTSEAFSYYLWLEAYYGRVTGDWKPLHDAWESMETFIIPGTKDQPTNSAYNPNSPATYIPEQPNADGYPSPLMNNVPVGQDPLAQELSSTYGTNEIYGMHWLLDVDNVYGFGFCGDGTDDAPAYINTYQRGARESVWETIPHPSCDDFTHGGPNGYLDLFTDDQNYAKQWRYTNAPDADARAVQVMFWAHEWAKEQGKENEIAGLMDKASKMGDYLRYAMFDKYFKKIGNCVGATSCPGGQGKDSAHYLLSWYYSWGGSLDTSSAWAWRIGSSSSHQGYQNVLAAYALSQVPELQPDSPTGVQDWATSFDRQLEFLQWLQSAEGGIAGGATNSWKGSYDTPPTGLSQFYGMYYDWQPVWNDPPSNNWFGFQVWNMERVAQLYYVTGDARAEAILDKWVPWAIQHTDVDADNGGQNFQVPSDLEWSGQPDTWTGTYTGNPNLHVQVVSYSQDVGVTAALAKTLMYYAKRSGDTTALATAEGLLDALLAHRDSIGIATPEQPSWDRLDDPWDGSEGLYVPPGWSGTMPNGDRIEPGATFLSIRSFYKNDPLWPQVEAHLNDPQNVPAPIVERHRFWAQVEIATAFAAHDELFGAGAP

SEQ ID NO: 5 - protein, 840 aa, Artificial sequence
  recombinant enzyme
MGHHHHHHAMGDSLKVTVGTANGKPGDTVTVPVTFADVAKMKNVGTCNFYLGYDASLLEVVSVDAGPIVKNAAVNFSSSASNGTISFLFLDNTITDELITADGVFANIKFKLKSVTAKTTTPVTFKDGGAFGDGTMSKIASVTKTNGSVTIDPTKGATPTNTATPTKSATATPTRPSVPRPHLQVDIGSTSGKAGSVVSVPITFTNVPKSGIYALSFRTNFDPQKVTVASIDAGSLIENASDFTTYYNNENGFASMTFEAPVDRARIIDSDGVFATINFKVSDSAKVGELYNITTNSAYTSFYYSGTDEIKNVVYNDGKIEVIASVPTNTPTNTPANTPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTKPPATTIPPSDDPNAIKIKVDTVNAKPGDTVNIPVRFSGIPSKGIANCDFVYSYDPNVLEIIEIKPGELIVDPNPDKSFDTAVYPDRKIIVFLFAEDSGTGAYAITKDGVFATIVAKVKSGAPNGLSVIKFVEVGGFANNDLVEQRTQFFDGGVNVGDIGSAGGLSAVQPNVSLGEVLDVSANRTAADGTVEWLIPTVTAAPGQTVTMPVVVKSSSLAVAGAQFKIQAATGVRYSSKTDGDAYGSGIVYNNSKYAFGQGAGRGIVAADDSVVLTLAYTVPADCAEGTYDVKWSDAFVSDTDGQNITSKVTLTDGAIIVK

SEQ ID NO: 6 - protein, 324 aa, Artificial sequence
  recombinant enzyme
MGSSHHHHHHTSPVIVYGDYNNDGNVDALDFAGLKKYIMAADHAYVKNLDVNLDNEVNAFDLAILKKYLLGMVSKLPSQDPNSVTGTVVELAPGIHAGFTGRAGGVSGEPYATLNLGDHVGDDPAAVAENRRRAALGFGISPDRVVWMNQVHGATAVTVTGSGQAGDVDAVVTPEAGLALAVLVADCLPLLVADAAAGVIGAAHAGRPGMAAGVVPALVAEMARHGARPERCVALLGPAICGRCYEVPRDLQDRVARTVPEARCTTAEGTPGLDIRAGVTAQLTNLGVTNITHDSRCTRESADLFSYRRDATTGRFAGYVWRVP

SEQ ID NO: 7 - protein, 705 aa, Artificial sequence
  recombinant enzyme
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTTSGSGSAGENLYFQGGSSHHHHHHTSPVIVYGDYNNDGNVDALDFAGLKKYIMAADHAYVKNLDVNLDNEVNAFDLAILKKYLLGMVSKLPSQDPNSVTGTVVELAPGIHAGFTGRAGGVSGEPYATLNLGDHVGDDPAAVAENRRRAALGFGISPDRVVWMNQVHGATAVTVTGSGQAGDVDAVVTPEAGLALAVLVADCLPLLVADAAAGVIGAAHAGRPGMAAGVVPALVAEMARHGARPERCVALLGPAICGRCYEVPRDLQDRVARTVPEARCTTAEGTPGLDIRAGVTAQLTNLGVTNITHDSRCTRESADLFSYRRDATTGRFAGYVWRVP

SEQ ID NO: 8 - DNA, 32 bp, Artificial sequence
  Single strand DNA oligonucleotide
ggtggtctcgagttattcttctttctcttcaa

SEQ ID NO: 9 - DNA, 42 bp, Artificial sequence
  Single strand DNA oligonucleotide
tatcgggtaccgcggccgcatttacaggttgacattggaagt

SEQ ID NO: 10 - DNA, 31 bp, Artificial sequence
  Single strand DNA oligonucleotide
tacgtggtaccgatgcaattacctcaatttt

SEQ ID NO: 11 - DNA, 2142 bp, Artificial sequence
  recombinant enzyme
atgggcagcagccatcaccatcatcaccacaagaatgcagattcctatgcgaaaaaacctcacatcagcgcattgaatgccccacaattggatcaacgctacaaaaacgagttcacgattggtgcggcagtagaaccttatcaactacaaaatgaaaaagacgtacaaatgctaaagcgccacttcaacagcattgttgccgagaacgtaatgaaaccgatcagcattcaacctgaggaaggaaaattcaattttgaacaagcggatcgaattgtgaagttcgctaaggcaaatggcatggatattcgcttccatacactcgtttggcacagccaagtacctcaatggttctttcttgacaaggaaggtaagccaatggttaatgaaacagatccagtgaaacgtgaacaaaataaacaactgctgttaaaacgacttgaaactcatattaaaacgatcgtcgagcggtacaaagatgacattaagtactgggacgttgtaaatgaggttgtgggggacgacggaaaactgcgcaactctccatggtatcaaatcgccggcatcgattatattaaagtggcattccaagcagctagaaaatatggcggagacaacattaagctttacatgaatgattacaatacagaagtcgaaccgaagcgaaccgctctttacaatttagtcaaacaactgaaagaagagggtgttccgatcgacggcatcggccatcaatcccacatccaaatcggctggccttctgaagcagaaatcgagaaaacgattaacatgttcgccgctctcggtttagacaaccaaatcactgagcttgatgtgagcatgtacggttggccgccgcgcgcttacccgacgtatgacgccattccaaaacaaaagtttttggatcaggcagcgcgctatgatcgtttgttcaaactgtatgaaaagttgagcgataaaattagcaacgtcaccttctggggcatcgccgacaatcatacgtggctcgacagccgtgcggatgtgtactatgacgccaacgggaatgttgtggttgacccgaacgctccgtacgcaaaagtggaaaaagggaaaggaaaagatgcgccgttcgtttttggaccggattacaaagtcaaacccgcatattgggctattatcgaccacaaatccgcgggtgaaaacctgtacttccagggtactagtcctgtaattgtatatggagattataacaatgatggaaatgttgatgcacttgattttgcaggcttaaagaaatatattatggctgctgaccatgcttatgtaaagaatttggatgttaatctcgacaatgaagtgaatgcatttgaccttgctattttgaaaaaatatctgcttggtatggtaagtaagctacctagccaggatccgaattcagtgacgggcaccgtggtcgagttggcccccgggatacacgccggattcaccggccgtgccggaggagtcagcggggagccgtacgcgaccctgaacctgggcgaccacgtgggtgacgaccctgcagcggtggcggagaaccggagacgggccgccctcgggttcgggatctcccccgaccgcgtggtgtggatgaaccaggtgcacggcgccaccgcggtgaccgtgaccggatccggccaggcgggggacgtcgacgcagtcgtcaccccggaagcaggcctcgccttggcggtgctggtggcggactgcctgcccctgctggtcgcggacgccgcagccggggtgatcggcgcggcgcacgcgggacgcccgggcatggcggcgggagtggtgcctgccctggtggcggagatggcccggcacggggcgcgccccgagcggtgtgttgccctcctggggcccgcgatctgcggccgctgctacgaggtgccccgcgacctgcaggacagggtggcccgcacggttccagaagcccgctgcacaaccgcggaaggcacaccaggactagacattcgagccggagtcaccgcacagttgacgaacttgggcgtgacgaatatcactcatgacagtcggtgtactcgggagagcgccgacttgttctcctaccgcagagacgcgaccaccggacggttcgccggatatgtctggagggtcccatga

SEQ ID NO: 12 - DNA, 1242 bp, Artificial sequence
  recombinant enzyme
atggcacaccatcaccatcaccatgcaccatcacccggcacaaagctcgttcctacatggggcgatacaaactgcgacggcgttgtaaatgttgctgacgtagtagttcttaacagattcctcaacgatcctacatattctaacattactgatcagggtaaggttaacgcagacgttgttgatcctcaggataagtccggcgcagcagttgatcctgcaggcgtaaagctcacagtagctgactctgaggcaatcctcaaggctatcgttgaactcatcacacttcctcaagcggtacccggcacgcagcccggcaccggcaccccggtcgagcggtacggcaaagtccaggtctgcggcacccagctctgcgacgagcacggcaacccggtccaactgcgcggcatgagcacccacggcatccagtggttcgaccactgcctgaccgacagctcgctggacgccctggcctacgactggaaggccgacatcatccgcctgtccatgtacatccaggaagacggctacgagaccaacccgcgcggcttcaccgaccggatgcaccagctcatcgacatggccacggcgcgcggcctgtacgtgatcgtggactggcacatcctcaccccgggcgatccccactacaacctggaccgggccaagaccttcttcgcggaaatcgcccagcgccacgccagcaagaccaacgtgctctacgagatcgccaacgaacccaacggagtgagctgggcctccatcaagagctacgccgaagaggtcatcccggtgatccgccagcgcgaccccgactcggtgatcatcgtgggcacccgcggctggtcgtcgctcggcgtctccgaaggctccggccccgccgagatcgcggccaacccggtcaacgcctccaacatcatgtacgccttccacttctacgcggcctcgcaccgcgacaactacctcaacgcgctgcgtgaggcctccgagctgttcccggtcttcgtcaccgagttcggcaccgagacctacaccggtgacggcgccaacgacttccagatggccgaccgctacatcgacctgatggcggaacggaagatcgggtggaccaagtggaactactcggacgacttccgttccggcgcggtcttccagccgggcacctgcgcgtccggcggcccgtggagcggttcgtcgctgaaggcgtccggacagtgggtgcggagcaagctccagtcctgactcgag

SEQ ID NO: 13 - DNA, 1365 bp, Artificial sequence
  recombinant enzyme
atgcaccatcaccatcaccacgatgtagtaattacgtcaaaccagacgggtactcacggcgggtacaactttgagtactggaaagacaccggaaacggaaccatggtcctcaaagacggtggtgcgttcagctgcgaatggagcaatatcaacaatattcttttccgtaaaggtttcaaatacgatgaaacaaagacacatgatcaacttggatacataacggtaacttattcctgcaactatcagccaaacggaaactcttatctgggagtctacggatggaccagcaatccgcttgtagagtattacatcatcgagagctggggaacctggagaccaccgggagcaacaccaaagggcactattaccgttgacggtggtacatacgagatatacgagaccaccagagttaaccagccttccatcaaaggtacagctactttccagcaatactggagtgtacgtacatcaaaacgtacaagcggaaccatatccgtaaccgaacactttaaagcctgggaacgtctgggtatgaaaatgggaaaaatgtatgaggttgctttggttgtagaaggataccagagcagcggaaaagccgacgtaaccagcatgacaattactgttggcaacgcaccgtcaacatcatcaccaccgggtccgacacctgaaccgactccaagaagtgctttttcaaaaatcgaagctgaggagtacaactccctcaagtcatcaaccattcagaccataggcacttccgacggaggaagcggtataggttatattgaaagcggtgactatctggtatttaacaaaataaactttggaaacggtgcaaactctttcaaggcaagggttgcatccggtgcggacacacccaccaatatccagttaagactcggaagcccgaccggtactcttataggaactcttacggtggcttccacaggcggttggaacaattacgaggaaaaatcctgcagcataaccaacactacaggacagcacgacttatatctggtattctcaggtcctgttaacattgactacttcatattcgactcgaaaggtgtaaatcctacacctacacctactagtactacaacaccaacgcctaaatttatatatggtgatgttgatggtaatggaagtgtaagaattaatgatgctgtcctaataagagactatgtattaggaaaaatcaatgaattcccatatgaatatggtatgcttgcagcagatgttgatggtaatggaagtataaaaattaatgatgctgttctagtaagagactacgtgttaggaaagatatttttattccctgttgaagagaaagaagaactcgagcaccaccaccaccaccactga

SEQ ID NO: 14 - DNA, 2220 bp, Artificial sequence
  recombinant enzyme
atggcacatcaccatcaccatcacgcagttgaaagcagttccacaggtctgggggatttaaatggtgacggaaatattaactcgtcggaccttcaggcgttaaagaggcatttgctcggtatatcaccgcttacgggagaggctcttttaagagcggatgtaaataggagcggcaaagtggattctactgactattcagtgctgaaaagatatatactccgcattattacagaggtacccggccacgactcggccgaggtgacggtccgggagatcgacccgaacaccagctcctacgaccaggccttcctggagcagtacgagaagatcaaggaccccgccagcggctacttccgcgaattcaacgggctcctggtcccctaccactcggtggagaccatgatcgtcgaggctccggaccacggccaccagaccacgtccgaggcgttcagctactacctgtggctggaggcgtactacggccgggtcaccggtgactggaagccgctccacgacgcctgggagtcgatggagaccttcatcatccccggcaccaaggaccagccgaccaactccgcctacaacccgaactccccggcgacctacatccccgagcagcccaacgctgacggctacccgtcgcctctcatgaacaacgtcccggtgggtcaagacccgctcgcccaggagctgagctccacctacgggaccaacgagatctacggcatgcactggctgctcgacgtggacaacgtctacggcttcgggttctgcggcgacggcaccgacgacgcccccgcctacatcaacacctaccagcgtggtgcgcgcgagtcggtgtgggagaccattccgcacccgtcctgcgacgacttcacgcacggcggccccaacggctacctggacctgttcaccgacgaccagaactacgccaagcagtggcgctacaccaacgcccccgacgctgacgcgcgggccgtccaggtgatgttctgggcgcacgaatgggccaaggagcagggcaaggagaacgagatcgcgggcctgatggacaaggcgtccaagatgggcgactacctccggtacgcgatgttcgacaagtacttcaagaagatcggcaactgcgtcggcgccacctcctgcccgggtggccaaggcaaggacagcgcgcactacctgctgtcctggtactactcctggggcggctcgctcgacacctcctctgcgtgggcgtggcgtatcggctccagctcctcgcaccagggctaccagaacgtgctcgctgcctacgcgctctcgcaggtgcccgaactgcagcctgactccccgaccggtgtccaggactgggccaccagcttcgaccgccagttggagttcctccagtggctgcagtccgctgaaggtggtatcgccggtggcgccaccaacagctggaagggaagctacgacaccccgccgaccggcctgtcgcagttctacggcatgtactacgactggcagccggtctggaacgacccgccgtccaacaactggttcggcttccaggtctggaacatggagcgcgtcgcccagctctactacgtgaccggcgacgcccgggccgaggccatcctcgacaagtgggtgccgtgggccatccagcacaccgacgtggacgccgacaacggcggccagaacttccaggtcccctccgacctggagtggtcgggccagcctgacacctggaccggcacctacaccggcaacccgaacctgcacgtccaggtcgtctcctacagccaggacgtcggtgtgaccgccgctctggccaagaccctgatgtactacgcgaagcgttcgggcgacaccaccgccctcgccaccgcggagggtctgctggacgccctgctggcccaccgggacagcatcggtatcgccacccccgagcagccgagctgggaccgtctggacgacccgtgggacggctccgagggcctgtacgtgccgccgggctggtcgggcaccatgcccaacggtgaccgcatcgagccgggcgcgaccttcctgtccatccgctcgttctacaagaacgacccgctgtggccgcaggtcgaggcacacctgaacgacccgcagaacgtcccggcgccgatcgtggagcgccaccgcttctgggctcaggtggaaatcgcgaccgcgttcgcagcccacgacgaactgttcggggccggagctccctga

SEQ ID NO: 15 - DNA, 2526 bp, Artificial sequence
  recombinant enzyme
atgggtcatcaccatcaccatcacgccatgggcgattctcttaaagttacagtaggaacagctaatggtaagcctggcgatacagtaacagttcctgttacatttgctgatgtagcaaagatgaaaaacgtaggaacatgtaatttctatcttggatatgatgcaagcctgttagaggtagtatcagtagatgcaggtccaatagttaagaatgcagcagttaacttctcaagcagtgcaagcaacggaacaatcagcttcctgttcttggataacacaattacagacgaattgataactgcagacggtgtgtttgcaaatattaagttcaaattaaagagtgtaacggctaaaactacaacaccagtaacatttaaagatggtggagcttttggtgacggaactatgtcaaagatagcttcagttactaagacaaacggtagtgtaacgatcgatccgaccaagggagcaacaccaacaaatacagctacgccgacaaaatcagctacggctacgcccaccaggccatcggtaccgcggccgcatttacaggttgacattggaagtactagtggaaaagcaggtagtgttgttagtgtacctataacatttactaatgtacctaaatcaggtatctatgctctaagttttagaacaaatttcgacccacaaaaggtaactgtagcaagtatagatgctggctcactgattgaaaatgcttctgattttactacttattataataatgaaaatggttttgcatcaatgacgtttgaagccccagttgatagagctagaatcatagatagtgatggtgtatttgcaaccattaactttaaagttagtgatagtgccaaagtaggtgaactttacaatattactactaatagtgcatatacttcattctattattctggaactgatgaaatcaaaaatgttgtttacaatgatggaaaaattgaggtaattgcatcggtaccgacaaacacaccgacaaacacaccggcaaatacaccggtatcaggcaatttgaaggttgaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgtaacaacaccacctgcaacaacaaaaccacctgcaacaacaaaaccacctgcaacaacaataccgccgtcagatgatccgaatgcaataaagattaaggtggacacagtaaatgcaaaaccgggagacacagtaaatatacctgtaagattcagtggtataccatccaagggaatagcaaactgtgactttgtatacagctatgacccgaatgtacttgagataatagagataaaaccgggagaattgatagttgacccgaatcctgacaagagctttgatactgcagtatatcctgacagaaagataatagtattcctgtttgcagaagacagcggaacaggagcgtatgcaataactaaagacggagtatttgctacgatagtagcgaaagtaaaatccggagcacctaacggactcagtgtaatcaaatttgtagaagtaggcggatttgcgaacaatgaccttgtagaacagaggacacagttctttgacggtggagtaaatgttggagatataggatccgccggtggtttatccgctgtgcagcctaatgttagtttaggcgaagtactggatgtttctgctaacagaaccgctgctgacggaacagttgaatggcttatcccaacagtaactgcagctccaggccagacggtcactatgcccgtagtagtcaagagttcaagtcttgcagttgctggtgcgcagttcaagatccaggcggcgacaggcgtacgttattcgtccaagacggacggtgacgcttacggttcaggcattgtgtacaataatagtaagtatgcttttggacagggtgcaggtagaggaatagttgcagctgatgattcggttgtgcttactcttgcatatacagttcccgctgattgtgctgaaggtacatatgatgtcaagtggtctgatgcgtttgtaagtgatacagacggacagaatatcacaagtaaggttactcttactgatggcgctatcattgttaagtaggat

SEQ ID NO: 16 - DNA, 975 bp, Artificial sequence
  recombinant enzyme
atgggcagcagccatcaccatcatcaccacactagtcctgtaattgtatatggagattataacaatgatggaaatgttgatgcacttgattttgcaggcttaaagaaatatattatggctgctgaccatgcttatgtaaagaatttggatgttaatctcgacaatgaagtgaatgcatttgaccttgctattttgaaaaaatatctgcttggtatggtaagtaagctacctagccaggatccgaattcagtgacgggcaccgtggtcgagttggcccccgggatacacgccggattcaccggccgtgccggaggagtcagcggggagccgtacgcgaccctgaacctgggcgaccacgtgggtgacgaccctgcagcggtggcggagaaccggagacgggccgccctcgggttcgggatctcccccgaccgcgtggtgtggatgaaccaggtgcacggcgccaccgcggtgaccgtgaccggatccggccaggcgggggacgtcgacgcagtcgtcaccccggaagcaggcctcgccttggcggtgctggtggcggactgcctgcccctgctggtcgcggacgccgcagccggggtgatcggcgcggcgcacgcgggacgcccgggcatggcggcgggagtggtgcctgccctggtggcggagatggcccggcacggggcgcgccccgagcggtgtgttgccctcctggggcccgcgatctgcggccgctgctacgaggtgccccgcgacctgcaggacagggtggcccgcacggttccagaagcccgctgcacaaccgcggaaggcacaccaggactagacattcgagccggagtcaccgcacagttgacgaacttgggcgtgacgaatatcactcatgacagtcggtgtactcgggagagcgccgacttgttctcctaccgcagagacgcgaccaccggacggttcgccggatatgtctggagggtcccatga

SEQ ID NO: 17 - DNA, 2118 bp, Artificial sequence
  recombinant enzyme
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactactagtggttctggttccgcgggtgaaaacctgtacttccagggtggcagcagccatcaccatcatcaccacactagtcctgtaattgtatatggagattataacaatgatggaaatgttgatgcacttgattttgcaggcttaaagaaatatattatggctgctgaccatgcttatgtaaagaatttggatgttaatctcgacaatgaagtgaatgcatttgaccttgctattttgaaaaaatatctgcttggtatggtaagtaagctacctagccaggatccgaattcagtgacgggcaccgtggtcgagttggcccccgggatacacgccggattcaccggccgtgccggaggagtcagcggggagccgtacgcgaccctgaacctgggcgaccacgtgggtgacgaccctgcagcggtggcggagaaccggagacgggccgccctcgggttcgggatctcccccgaccgcgtggtgtggatgaaccaggtgcacggcgccaccgcggtgaccgtgaccggatccggccaggcgggggacgtcgacgcagtcgtcaccccggaagcaggcctcgccttggcggtgctggtggcggactgcctgcccctgctggtcgcggacgccgcagccggggtgatcggcgcggcgcacgcgggacgcccgggcatggcggcgggagtggtgcctgccctggtggcggagatggcccggcacggggcgcgccccgagcggtgtgttgccctcctggggcccgcgatctgcggccgctgctacgaggtgccccgcgacctgcaggacagggtggcccgcacggttccagaagcccgctgcacaaccgcggaaggcacaccaggactagacattcgagccggagtcaccgcacagttgacgaacttgggcgtgacgaatatcactcatgacagtcggtgtactcgggagagcgccgacttgttctcctaccgcagagacgcgaccaccggacggttcgccggatatgtctggagggtcccatga

SEQ ID NO: 18 - DNA, 56 bp, Artificial sequence
  Single strand DNA oligonucleotide
attatgcatatgcaccatcaccatcaccacgatgtagtaattacgtcaaaccagac

SEQ ID NO: 19 - DNA, 46 bp, Artificial sequence
  Single strand DNA oligonucleotide
attctactcgagattatcactagtaggtgtaggtgtaggatttaca

SEQ ID NO: 20 - DNA, 33 bp, Artificial sequence
  Single strand DNA oligonucleotide
ctacaactagtactacaacaccaacgcctaaat

SEQ ID NO: 21 - DNA, 23 bp, Artificial sequence
  Single strand DNA oligonucleotide
tataccatggtgacgggcaccgt

SEQ ID NO: 22 - DNA, 23 bp, Artificial sequence
  Single strand DNA oligonucleotide
ggtgctcgagtgggaccctccag

SEQ ID NO: 23 - DNA, 49 bp, Artificial sequence
  Single strand DNA oligonucleotide
cggttcgccggatatgtctggagggtcccaactagtcctgtaattgtat

SEQ ID NO: 24 - DNA, 49 bp, Artificial sequence
  Single strand DNA oligonucleotide
ggtggcagcagcctaggttaattaagctgcttaaggtagcttacttacc

SEQ ID NO: 25 - DNA, 60 bp, Artificial sequence
  Single strand DNA oligonucleotide
atgggcagcagccatcaccatcatcaccacaagaatgcagattcctatgcgaaaaaacct

SEQ ID NO: 26 - DNA, 60 bp, Artificial sequence
  Single strand DNA oligonucleotide
accctggaagtacaggttttcacccgcggatttgtggtcgataatagcccaatatgcggg

SEQ ID NO: 27 - DNA, 50 bp, Artificial sequence
  Single strand DNA oligonucleotide
tataccatggcacatcaccatcaccatcacgcagttgaaagcagttccac

SEQ ID NO: 28 - DNA, 50 bp, Artificial sequence
  Single strand DNA oligonucleotide
gtcacctcggccgagtcgtggccgggtacctctgtaataatgcggagtat